(19) United States
(12) Patent Application Publication
(10) Pub. No.: US 2003/0084466 A1
(43) Pub. Date: May 1, 2003

(54) System and method for controlling male fertility

(76) Inventors: Peter Blume-Jensen, Cambridge, MA (US); Tony Hunter, Del Mar, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/192,867

(22) Filed: Jul. 8, 2002

Related U.S. Application Data
(63) Continuation of application No. PCT/US01/00573, filed on Jan. 8, 2001.
(60) Provisional application No. 60/175,625, filed on Jan. 11, 2000.

(51) Int. Cl. A01K 67/027

(52) U.S. Cl. 800/18; 800/21

(57) ABSTRACT

The invention described herein relates to modulation of fertility in male mammals. A genetically altered mouse having a mutation in the Kit/stem cell factor receptor protein is described. In addition, antibodies that specifically bind to a Kit/stem cell factor receptor are described. Methods to diagnose Kit-mediated fertility defects; methods to identify agents that modulate Kit-mediated activation of phosphatidylinositol 3′-kinase and thereby modulate spermatogenesis; and pharmaceuticals that modulate spermatogenesis by effecting Kit-mediated activation of phosphatidylinositol 3′-kinase are also described.

### CROSS-REFERENCE TO RELATED APPLICATIONS

This application is a continuation of international application number PCT/US01/00573, and claims the benefit of priority of international application number PCT/US01/00573 having international filing date of Jan. 8, 2001, designating the United States of America and published in English, which claims the benefit of priority of U.S. provisional patent application No. 60/175,625, filed Jan. 11, 2000; both of which are hereby expressly incorporated by reference in their entireties.

### GOVERNMENTAL INTEREST

The invention disclosed was made with Government support under NIH Grant No. R01 CA39780 and R01 CA82683.

### FIELD OF THE INVENTION

The present invention relates generally to the field of human fertility. More particularly, the invention relates to the discovery that the interaction between phosphotidylinositol 3′-kinase and the intracellular domain of a Kit/stem cell factor receptor modulates fertility in male mammals.

### BACKGROUND OF THE INVENTION

The c-kit-encoded transmembrane tyrosine kinase receptor for stem cell factor (Kit/SCF-R) is required for normal hematopoiesis, melanogenesis and gametogenesis. (Besmer, et al., Dev. Suppl., 125-37 (1993); Galli et al., Adv Immunol, 55:1-96 (1994); and Lyman and Jacobsen, Blood, 91:1101-34 (1998)). Kit/SCF-R, also called the “Kit” receptor, is encoded by the murine dominant white-spotting (W) locus in which a number of naturally occurring mutations have been described. In mice heterozygous for the W mutation, for example, hematopoiesis and melanogenesis are most often affected, while decreased fertility is only rarely observed. (Besmer, et al., Dev. Suppl., 125-37 (1993) and Galli et al., Adv Immunol, 55:1-96 (1994)). The loss-of-function W mutations negatively affect all signaling pathways from the receptor. (Besmer, et al., Dev. Suppl., 125-37 (1993); Galli et al., Adv Immunol, 55:1-96 (1994); and Lyman and Jacobsen, Blood, 91:1101-34 (1998)). Further, antibodies that block binding of stem cell factor (SCF) to the Kit receptor have been shown to perturb hematopoiesis, melanogenesis and gametogenesis. Ogawa et al., Development, 117(3):1089-98 (1993); Nishikawa et al., EMBO J, 10(8):2111-8 (1991); and Yoshinaga et al., Development, 113:689-699 (1991).

The intracellular region of the mammalian Kit receptor has five distinct domains. In the human Kit receptor, for example, the juxtamembrane region is located at approximately amino acid positions 544-594, a first kinase domain is located at approximately amino acid positions 595-686, a kinase insert domain is located at approximately amino acid positions 687-763, a second kinase domain is located at approximately amino acid positions 764-917, and a carboxy terminal domain is located at approximately amino acid positions 918-976. In response to binding of a bivalent ligand, SCF, the Kit receptor undergoes dimerization and the concomitant kinase activation results in tyrosine autophosphorylation in the intracellular region of the receptor. Specific tyrosine phosphorylation sites, in turn, create specific binding sites for intracellular signaling molecules, which bind through their SH2 domains.

One such molecule is phosphatidylinositol 3′-kinase (PI 3′-kinase), which consists of a p85 regulatory subunit and a p110 catalytic subunit. Investigators have solved the crystal structure of the amino terminal SH2 domain of the p85 α subunit of PI 3′-kinase, alone and in complex with phosphopeptides bearing a consensus binding motif. (Nolte et al., Nature Struct. Biol., 3:364-373 (1996). Further, investigators have shown that polyclonal antibodies that recognize epitopes within amino acid residues 719-735 of the human Kit receptor partially inhibit the binding of PI 3′ kinase to the Kit receptor. (Levy et al., Proc. Natl. Acad. Sci. USA 89:678-682 (1992)). These data present a model in which the amino terminal SH2 domain in p85 specifically binds to the Kit receptor by interacting with a consensus sequence, which includes a phosphorylated tyrosine residue at position 719 (in mice) and a phosphorylated tyrosine residue at position 721 (in humans).

Both binding and tyrosine phosphorylation of p85 induce a conformational change in the p85 regulatory subunit, which results in allosteric activation of the p110 catalytic subunit of PI 3′-kinase. Once activated, PI 3′-kinase induces an elaborate signal transduction cascade that results in activatation of several down stream signaling molecules, including a serine/threonine kinase, called Akt, which is involved in cell proliferation and cell survival. At present, the understanding of Kit/SCF-R-induced signaling pathways and the control of developmental processes in the intact animal is in its infancy.

### SUMMARY OF THE INVENTION

The Kit/stem cell factor receptor kinase insert region was mutated through the use of the Cre-loxP system (Metzger and Feil, Curr. Opin. Biotechnol., 10:470-6 (1999)) to replace the codon for Tyrosine (Tyr) at amino acid position 719, the PI 3′-kinase binding site in Kit/SCF-R, with phenylalanine (Phe) in the genome of mice by homologous recombination.

Homozygous (Y719F/Y719F) genetically altered mice were found to be viable. However, the mutation completely disrupted PI 3′-kinase binding to Kit/SCF-R and reduced SCF-induced PI 3′-kinase-dependent activation of Akt by approximately 90%. Surprisingly, the mutation induced a gender-and tissue-specific defect. Although there were no hematopoietic or pigmentation defects in homozygous mutant mice, males were found to be sterile due to a block in spermatogenesis, characterized by initially decreased cell proliferation and subsequent extensive apoptosis occurring at the spermatogonial stem cell level. In contrast, female Y719F/Y719F homozygotes were fully fertile.

Accordingly, one embodiment of the invention includes a homozygous genetically altered mouse having a Y719F mutation in both alleles of the Kit/stem cell factor receptor.

Another embodiment is compounds that specifically interfere with the binding of phosphatidylinositol 3′-kinase to the Kit/stem cell factor receptor. These compounds can include peptidomimetics, antibodies, peptides, chemicals, or other compounds. This embodiment includes factors that block the phosphorylation of tyrosine residues within the kinase insert domain of the Kit/SCF-R. Such factors include those compounds that bind to the tyrosine 719 (mice) or tyrosine 721 (human) positions of the Kit/SCF-R.

Yet another embodiment is methods of diagnosing Kit-mediated fertility defects and methods to identify agents that modulate Kit-mediated activation of phosphatidylinositol 3′-kinase and thereby modulate spermatogenesis are contemplated.

Still further, embodiments of the invention include pharmaceuticals that modulate spermatogenesis by effecting Kit-mediated activation of phosphatidylinositol 3′-kinase.

### BRIEF DESCRIPTION OF THE DRAWINGS

FIG. 1 Disruption of SCF-induced PI 3′-kinase signaling in mice by mutation of the PI 3′-kinase-binding site in Kit/SCF-R. Panel (a) shows a targeting construct containing the Y719F mutation in Kit exon 15 and a loxP-flanked neo marker in intron 14 that was used to target Prm-Cre ES cells (step I). Cre-mediated neo excision in the germ cells of male chimeras leaves only an extra 87 bp behind in the intron together with the Y719F mutation for germ line transmission (step II). Panel (b) shows the results from a Southern blot of XbaI digests. Probe A identifies a 7 kb wild type fragment and an 8.7 kb gene-targeted fragment. Panel (c) shows the results from PCR genotyping of the knock-out mice, which reveals the loxP site. The PCR makes use of an EcoRI site introduced at the Y719F mutation. Panels (d and e) show the results of Kit immunoprecipitations from isolated mast cells. Immunoprecipitates were probed for Kit and reprobed for phosphotyrosine. Panels (f and g) show the results of immunoprecipitations of the p85 subunit of PI 3′-kinase. These immunoprecipitates were probed for p85 and reprobed for phosphotyrosine. Panel (h) shows the results of Akt in vitro kinase assays using histone H2B as a substrate. “F/+” indicates Y719F/+heterozygous mice and “F/F” indicates Y719F/Y719F homozygous mice.

FIG. 2 Testes, ovaries and hair follicles of adult (4 months old) mice. Panel (a) shows the testes from +/+ (left) and littermate Y719F/Y719F (right) mice. Panels (b-i) are sections of +/+ and Y719F/Y719F testes. These sections show spermatogonial stem cells (closed arrow heads), and Sertoli cells (open arrow heads). “I” indicates primary spermatocytes, “II” indicates secondary spermatocytes, “S” indicates spermatids, and “Sp” indicates spermatozoa. The capsules of some individual seminiferous tubules are visible in panels (d-e and h-i). Note the outer layer of Kit-positive cells in +/+ testes, shown in panel (d), and presence of cells within the capsules of seminiferous tubules from Y719F/Y719F mutant mice, shown in panel (i). The Kit staining in panels (d and h) was performed with CY-3-conjugated secondary antibodies. Panels (e and i) are DAPI stainings of the same sections. Panels (j-k) show sections of ovaries revealing oocytes (closed arrow heads) and ruptured ovarian follicles (open arrow heads). Panels (l-m) are sections of hair follicles in lumbar skin. The arrow heads indicate melanocytes. “H & E” indicates hematoxylin/eosin staining. Bars are 100 μm.

FIG. 3 Primordial germ cells (PGC) in genital ridges and cell proliferation, apoptosis, and Kit positive cells in parallel sections of testes from 8 day old (P8) pups. Panels (a-d) show embryos at 13.5 dpc. These embryos were genotyped and their genital ridges were dissected out. PGC were visualized by alkaline phosphatase staining of whole mounts of male genital ridges. Panels (e-h) show BrdU labeling in sections of testes from P8 pups in vivo. These sections reveal proliferating cells. Counter staining was performed with hematoxylin. Panels (i-l) show the results of a TUNEL in situ assay performed on parallel sections. Cells were visualized with nitroblue-tetrazolium/X-phosphate. No apoptotic cells were revealed at this stage. Panels (m-p) are sections that reveal the presence of Kit-positive cells in the outer cell layer within the seminiferous tubules and scattered throughout the interstitium; note, fewer Kit-positive cells are seen in the mutant testis. Panels (q-t) show the results of DAPI staining of the same sections as shown in panels (m-p).

FIG. 4 Cell proliferation, apoptosis, and Kit positive cells in parallel sections of testes from 10 day old (P10) pups. Panels (a-d) show BrdU labeling in sections of testes from P10 pups in vivo. These sections reveal proliferating cells. Counter staining was performed with hematoxylin. Panels (e-h) show the results of a TUNEL in situ assay performed on parallel sections. Cells were visualized with nitroblue-tetrazolium/X-phosphate. Dark apoptotic cells in the mutant testis were reveald at this stage, see panels (g-h). Panels (i-l) are sections that reveal the presence of Kit-positive cells in the outer cell layer of seminiferous tubules and scattered throughout the interstitium; note, fewer Kit-positive cells are seen in the mutant testis. Panels (m-p) show the results of DAPI staining of the same sections as shown in panels (i-l).

### DETAILED DESCRIPTION OF THE INVENTION

Embodiments of the present invention relate to systems and methods for modulating fertility in male mammals. These embodiments result from the discovery that modifications of the amino acid sequence of the kinase insert domain of the Kit/stem cell factor receptor (“Kit/SCF-R” or “Kit”) resulted in sterility of male mice, but not other obvious phenotypic alterations. Specifically, mutations of the Kit/SCF-R have been identified that inhibit the interaction of the Kit/SCF-R and phosphatidylinositol-3 Kinase (“PI 3′-kinase”).

In one embodiment, the invention includes systems and methods for inhibiting the interaction of Kit/SCF-R and PI 3′-kinase by altering the phosphorylation pattern of the kinase insert domain of the Kit/SCF-R. More specifically, embodiments include modification of one or more tyrosine residues within the kinase insert domain such that PI 3′-kinase no longer binds and interacts with the Kit receptor. Modification includes mutation, substitution or alteration of the residue by any manner that inhibits PI 3′-kinase binding.

It should realized that complete inhibition of binding is not always the desired result. For example, compounds that partially block binding of PI 3′-kinase could be used to lessen the fertility of mammals, such as mice, without completely abrogating the ability of the mammal to breed. Such compounds could include fragments of the kinase insert region that are used to saturate PI 3′-kinase binding, and thus lower the amount of PI 3′-kinase available to bind with the receptor.

Other embodiments include factors that increase the binding of PI 3′-kinase to Kit/SCF-R in a mammal for treating fertility disorders. Such compounds can be given to a mammal, such as a human, in order to increase the fertility of the mammal.

While specific examples are given for mice and humans, systems and methods that involve other mammals are also contemplated. Thus, reference to a “kinase insert region” refers to such a region in any mammal. Similarly, reference to the “Kit/SCF-R” refers to that receptor in any mammal. As discussed above the kinase insert region runs from approximately amino acid number 687-763 in humans.

One embodiment of the invention relates to sterile genetically altered mice produced by replacement of the tyrosine residue at position 719 of the murine Kit/stem cell factor receptor with a phenylalanine residue. However, it should be realized that this invention is not limited to only this particular mutation. Any mutation in the intracellular domain of the Kit/stem cell factor receptor that results specifically in male sterility is contemplated. For example, other mutations of amino acid sequences within the kinase insert region of the intracellular domain that specifically affect male fertility are contemplated.

Moreover, embodiments of the invention are not limited to murine mammals. Human males express a Kit/stem cell factor receptor, and provide a similar tyrosine residue at amino acid position 721. Methods for providing human male contraception via specifically inhibiting Kit-induced PI 3′-kinase activation are also contemplated. Moreover, compounds that block or otherwise inhibit the effect of the kinase insert region, or tyrosine 721, are anticipated to result in either temporary or permanent sterility of male humans.

As described below, genetic differences, mutations or polymorphisms that occur in the gene sequence encoding the intracellular domain of the Kit/stem cell factor receptor contribute to sterility in male mammals. In the most prevalent form of Kit, a tyrosine residue is present at amino acid position 719 (in mice) or amino acid position 721 (in humans) given by the sequences provided in the sequence listing (SEQ ID. Nos. 2 and 4), respectively. These wild type forms of Kit protein, or polynucleotides encoding these wild type forms of Kit (i.e., Kit proteins having a tyrosine residue at amino acid position 719 (in mice) or amino acid position 721 (in humans)), are referred to throughout this disclosure as “Tyr719” or “Y719” and “Tyr721” or “Y721,” respectively. The cDNA encoding the murine Kit protein is provided in the appended Sequence Listing (SEQ ID No. 1) and the cDNA encoding the human kit protein is provided in the Sequence Listing (SEQ ID No. 3).

A single amino acid mutation in murine Kit, called “Y719F”, resulted in a male sterile phenotype. This form of murine Kit receptor was characterized by a substitution of a phenylalanine residue in place of the tyrosine residue that is ordinarily present at amino acid position 719. In some contexts, the term “Tyr719Phe” or “Y719F” refers to a mutation in a polynucleotide encoding the murine Kit receptor (in which case the mutation is with reference to the codon encoding amino acid position 719 of the murine Kit receptor), or to the murine Kit receptor itself (in which case the mutation is with reference to amino acid position 719 of the murine Kit receptor polypeptide sequence given by (SEQ ID. No. 2). In a more general sense, mutant forms of the murine Kit protein and genes encoding the Kit receptor are referred to as “Y719X”, wherein “X” denotes any amino acid residue except tyrosine. Accordingly, the Y719F is a type of mutant Kit receptor that is embodied by the class of mutant, murine Kit receptors designated Y719X.

In a similar manner, mutant or polymorphic human Kit proteins and the genes encoding these receptors are referred to throughout this disclosure as “Y721X”, wherein “X” denotes any amino acid residue except tyrosine, with reference to amino acid position 721 of the murine Kit receptor polypeptide sequence given by (SEQ ID. No. 4). Moreover, one specific human mutant (i.e. the polypeptide or polynucleotide encoding the polypeptide) is termed herein “Y721F” with reference to the polypeptide sequence provided in the sequence listing (SEQ ID. No. 4).

To further investigate the role of Kit in signal transduction in vivo, Kit -induced PI 3′-kinase activation was disrupted in mice. Examples 1-5 below describe the creation of a genetically altered mouse having the Y719F point mutation introduced by homologous recombination.

Approaches to Producing Genetically Altered Mice

As discussed below, mice that were homologous for the Y719F mutation were found to be sterile, but did not exhibit any other detectable phenotypic differences from wild-type mice. Thus, these mice provide an advantageous model for sterility testing in a mammal.

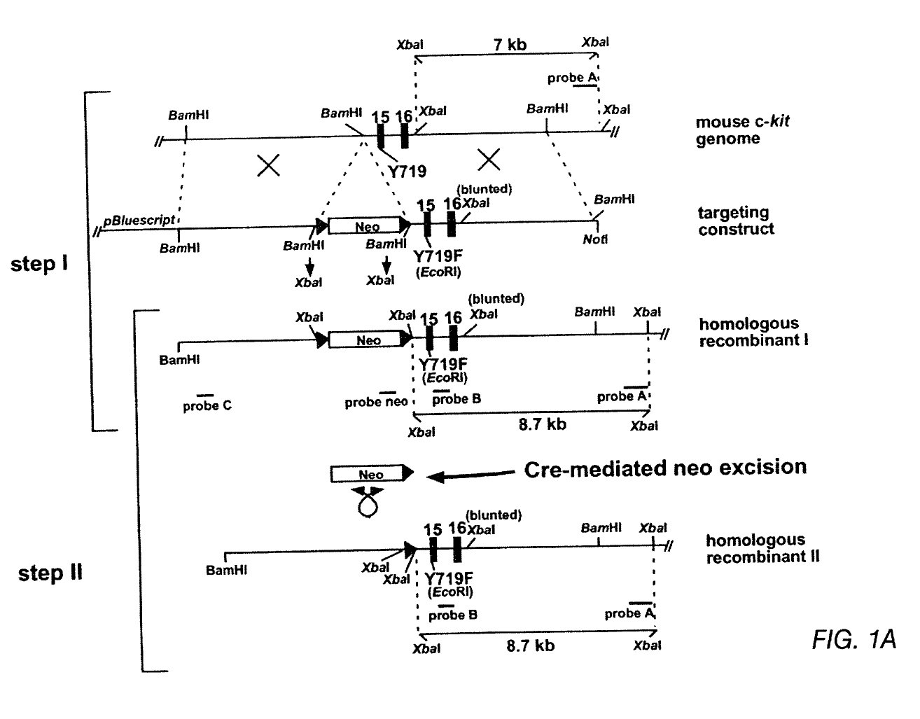

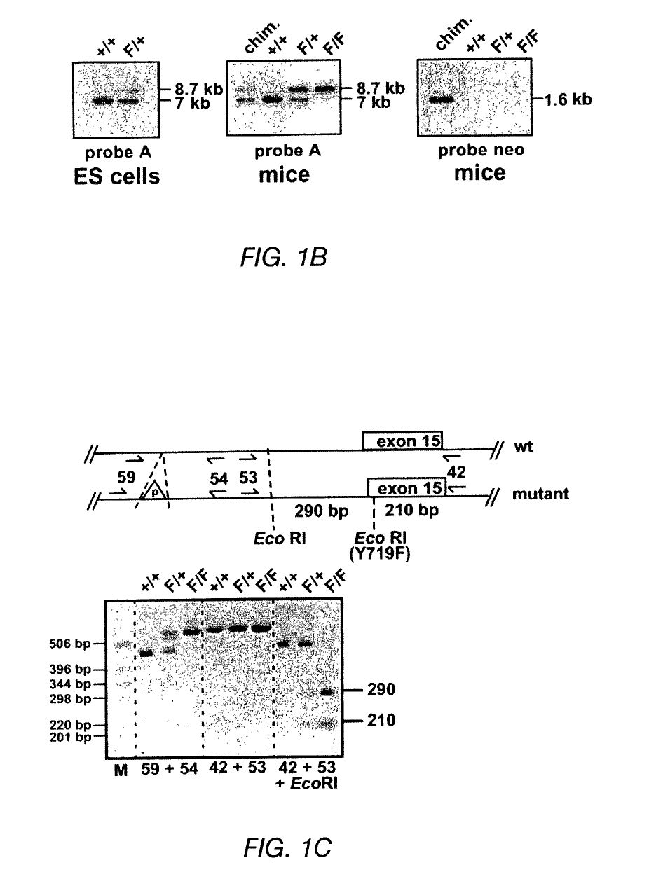

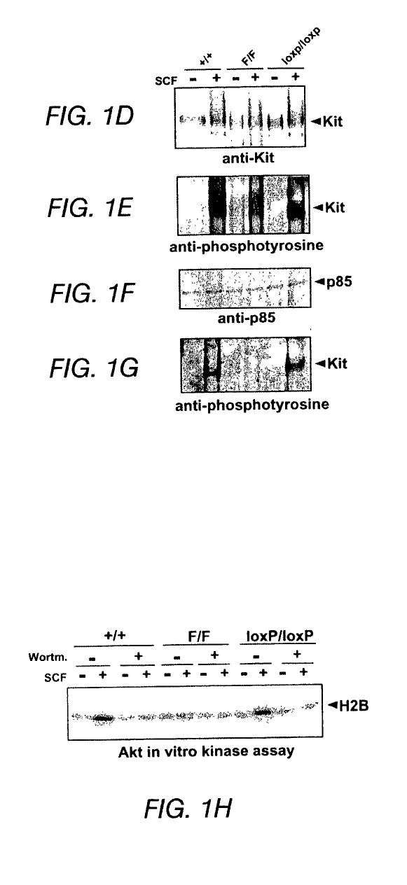

Briefly, a mutation of the single PI 3′-kinase-binding site in the Kit/SCF-R, Y719F, was generated by gene targeting in embryonic stem (ES) cells with 13.6 kb of homologous sequence containing the desired point mutation in Kit exon 15 and a loxP-flanked (‘floxed’) neo marker in intron 14. (See FIG. 1(a) and EXAMPLE 1). The introduced loxP sites were used for subsequent Cre-mediated excision of the neo gene to avoid potential transcriptional interference. The ES cells used in these experiments contained a Cre recombinase transgene under control of the protamine-1 (Prm1) promoter, which is active in male haploid germ cells, so that Cre-mediated excision could take place. (O'Gorman et al., Proc. Natl. Acad. Sci. USA, 94:14602-7 (1997)).

Accordingly, while male chimeric mice derived from targeted ES cells contained the neo gene in somatic tissues, it was found that the neo gene was absent in all mutant offspring. (FIGS. 1(a-c)). As a consequence, only one loxP site and surrounding Xba linker sequences, totaling 87 bp, were left behind in intron 14 of Kit, upstream of the desired point mutation. Both the loxP site and the point mutation were detected by PCR genotyping, since a diagnostic EcoRI restriction site along with the Y719F codon had been introduced. (FIG. 1(c) and EXAMPLE 2). Homozygous Y719F/Y719F and heterozygous Y719F/+mice were born at the expected Mendelian ratios in the F₃ generation, had normal body weights, and good general health.

In the section below, several approaches are disclosed to manufacture antibodies that specifically recognize wild type and mutant forms of Tyr719 or Tyr721 (i.e. wild type Y719 or Y721 Kit receptors and mutant Y719X or Y721X Kit receptors).

Approaches to Producing Antibodies

Depending on the context, the term “antibodies” can encompass polyclonal, monoclonal, chimeric, single chain, Fab fragments and fragments produced by a Fab expression library. Embodiments of the invention include antibodies that recognize the kinase insert region of the Kit receptor and specifically inhibit spermatogenesis. Also included are antibodies that specifically bind to murine mutant Kit receptors having the general structural formula Y719X, wherein “X” denotes an amino acid other than tyrosine. In one embodiment, for example, an antibody to Y719F is contemplated and can be used to identify mice having the Y719F mutation. More desirable antibodies of the invention, however, are diagnostic for fertility defects by binding and identifying human mutant Kit receptors. Additional desirable antibodies bind to the kinase insert region and block spermatogenesis as a mechanism for providing contraception.

In one embodiment, the antibodies bind to human Kit receptors having the general structural formula Y721X, wherein “X” denotes an amino acid other than tyrosine at amino acid position 721. These antibodies can be used, for example, in diagnostic devices and methods, which identify human males that suffer from Kit-mediated fertility defects.

Additional embodiments of the invention include antibodies directed to Tyr719 or Tyr721. In an effort to identify the PI 3′ kinase binding domain of the human Kit receptor, investigators have manufactured polyclonal antibodies that recognize epitopes within amino acid residues 719-735 of the human Kit receptor (a region referred to as the “KI domain”). (Levy et al., Proc. Natl. Acad. Sci. USA 89:678-682 (1992)). These antibodies were shown to only partially inhibit the binding of PI 3′ kinase to the Kit receptor. The inventors contemplate that monoclonal and polyclonal antibodies that more efficiently block binding of PI 3′ kinase to the murine and human Kit receptor can be valuable biotechnological tools. More desirable, however, these antibodies can be used inhibit spermatogenesis, or as templates to generate and identify other compounds (e.g., peptidomimetics) that inhibit spermatogenesis.

For the production of antibodies according to aspects of the invention, various hosts including humans, sheep, goats, rabbits, rats, mice, etc are immunized by injection with a selected antigen in combination with an adjuvant. Depending on the host species, the various adjuvants are used individually or in combination. Adjuvants that are used with embodiments of the invention include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guerin) and Corynebacterium parvum.

Monoclonal antibodies are prepared using any technique that provides for the production of antibody molecules by continuous cell lines in culture. These include but are not limited to the hybridoma technique originally described by Koehler and Milstein (Nature 256:495-497 (1975), the human B-cell hybridoma technique (Kosbor et al. Immunol Today 4:72 (1983); Cote et al Proc Natl Acad Sci 80:2026-2030 (1983), and the EBV-hybridoma technique Cole et al. Monoclonal Antibodies and Cancer Therapy, Alan R. Liss Inc, New York N.Y., pp 77-96 (1985). In addition, techniques developed for the production of “chimeric antibodies”, the splicing of mouse antibody genes to human antibody genes to obtain a molecule with appropriate antigen specificity and biological activity are used. (Morrison et al. Proc Nati Acad Sci 81:6851-6855 (1984); Neuberger et al. Nature 312:604-608(1984); Takeda et al. Nature 314:452-454(1985). Alternatively, techniques described for the production of single chain antibodies (U.S. Pat. No. 4,946,778) are adapted to produce specific single chain antibodies.

Antibody fragments that contain specific binding sites for the selected antigen can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed to allow rapid and easy identification of monoclonal Fab fragments with the desired specificity. (Huse W. D. et al. Science 256:1275-1281 (1989)).

By one approach, monoclonal antibodies to a selected antigen (e.g. amino acid residues 687-763, 700-735, more specifically 719-735, even more specifically any immunogenic region near the tyrosine 719 (mice) or 721 (human) residue or other regions of the intracellular domain of the Kit receptor) are made as follows:

A polypeptide consisting of amino acid residues 700-735 of human Kit receptor, for example, is manufactured by recombinant techniques or by methods of solid-phase peptide synthesis. Next, a mouse is repetitively inoculated with a few micrograms of the selected antigen in combination with an adjuvant. After several weeks of inoculation, the mouse is sacrificed, and the antibody producing cells of the spleen are isolated. The spleen cells are fused in the presence of polyethylene glycol with mouse myeloma cells, and the excess un-fused cells are destroyed by growth of the system on selective media comprising aminopterin (HAT media).

The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, N.Y. Section 21-2.

Polyclonal antiserum containing antibodies directed to a selected antigen (e.g. amino acid residues 700-735 or other regions of the intracellular domain of the Kit receptor) can be prepared by immunizing subjects with the antigen in combination with an adjuvant, combination of adjuvants, and a carrier. Because small molecules tend to be less immunogenic, the use of carriers and more than one adjuvant may be required. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971).

Booster injections are given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980).

Additionally, humans can be provided the selected antigen (e.g. amino acid residues 700-735 or other immunogenic regions of the intracellular domain of the Kit receptor) in the form of a pharmaceutical with or without a carrier or adjuvant fit for human vaccination so as to induce a preliminary immune response to the antigen and concomitantly block PI 3′-kinase binding upon phosphorylation of the peptide.

The peptide can be covalently linked to a phosphate prior to introduction or phosphorylation can be allowed to occur in vivo. That is, a selected antigen comprising regions of the intracellular domain of the Kit receptor can be administered to a subject in need in combination with a carrier and/or an adjuvant so as to activate the antigen presenting cells of the subject to induce a primary immune response, therein. An additional benefit from such a pharmaceutical is that the peptide, upon phosphorylation, can effectively bind to PI 3′ kinase and thereby prevent PI 3′ kinase from binding to endogenous Kit receptor. Furthermore, nucleic acids encoding the selected antigens discussed above can be administered by themselves or with the corresponding protein. These nucleic acids can be administered “naked” or can be incorporated into vectors. Treatment protocols can include, for example, identifying a subject in need of an inhibition of spermatogenesis (e.g., an individual preparing to undergo cancer therapy) and providing to said individual a therapeutically effective amount of a pharmaceutical comprising a peptide having amino acid residues 700-735 or other regions of the intracellular domain of the Kit receptor.

Many of the antibodies of the invention can be used in diagnostic methods to identify individuals suffering from Kit-mediated fertility defects. In the disclosure below, several of the diagnostic embodiments of the invention are described.

Diagnostic Embodiments

Generally, the diagnostics of the invention can be classified according to whether the embodiment is a nucleic acid or protein-based assay. These assays identify and distinguish fertility defects by detecting the presence of mutations in the kinase insert region of the Kit receptor. In one embodiment, the mutation is a dephosphorylation of a tyrosine residue within the kinase insert region. Other mutations include substitutions or alterations in the amino acid sequence of the Kit receptor. Examples of such substitutions or alterations include Y719X or Y721X, wherein the tyrosine at postion 719 (mice) or 721 (human) are replaced with other amino acids.

Other diagnostic techniques involve identification of compounds that interfere with spermatogenesis through the Kit-receptor/PI 3′-kinase mechanism. The identification of such compounds would be diagnostic for a fertility defect in a mammal.

Some of the diagnostic embodiments focus on the detection of the presence or absence of a tyrosine amino acid residue in a protein sample. While and other diagnostic embodiments focus on the detection of a polynucleotide encoding a tyrosine residue within the kinase insert region in a nucleic acid sample.

Additionally, the manufacture of kits that incorporate the reagents and methods described in the following embodiments so as to allow for the rapid detection and identification of fertility defects are contemplated. The diagnostic kits can include a nucleic acid probe or an antibody or combinations thereof, which specifically detect a Kit receptor mutation, such as the Y719X or Y721X mutation. The detection component of these kits will typically be supplied in combination with one or more of the following reagents. A support capable of absorbing or otherwise binding DNA, RNA, or protein will often be supplied. Available supports include membranes of nitrocellulose, nylon or derivatized nylon that can be characterized by bearing an array of positively charged substituents. One or more restriction enzymes, control reagents, buffers, amplification enzymes, and non-human polynucleotides like calf-thymus or salmon-sperm DNA can be supplied in these kits.

Useful nucleic acid-based diagnostic techniques include, but are not limited to, direct DNA sequencing, Southern Blot analysis, single-stranded confirmation analysis (SSCA), RNase protection assay, dot blot analysis, nucleic acid amplification, and combinations of these approaches. The starting point for these analysis is isolated or purified DNA from a biological sample. Most simply, blood is obtained from a subject to be tested. Additionally, it is contemplated that tissue biopsies would provide a good sample source. DNA is extracted from the sample and can be amplified by a DNA amplification technique such as the Polymerase Chain Reaction (PCR) using primers that correspond to regions flanking DNA that encodes amino acid residues within the kinase insert region. Examples include DNA primers that amplify amino acid 719 (in mice) or 721 (in humans).

Once a sufficient amount of DNA is obtained from an individual to be tested, several methods can be used to detect a Kit receptor polymorphism in the kinase insert region. Direct DNA sequencing, either manual sequencing or automated fluorescent sequencing can detect such sequence variations. Another approach is the single-stranded confirmation polymorphism assay (SSCA) (Orita et al., Proc. Natl. Acad. Sci. USA 86:2776-2770 (1989). This method, however, does not detect all sequence changes, especially if the DNA fragment size is greater than 200 base pairs, but can be optimized to detect most DNA sequence variation.

The reduced detection sensitivity is a disadvantage, but the increased throughput possible with SSCA makes it an attractive, viable alternative to direct sequencing for mutation detection. The fragments that have shifted mobility on SSCA gels are then sequenced to determine the exact nature of the DNA sequence variation. Other approaches based on the detection of mismatches between the two complimentary DNA strands include clamped denaturing gel electrophoresis (CDGE) (Sheffield et al., Am. J. Hum. Genet. 49:699-706 (1991)), heteroduplex analysis (HA) (White et al., Genomics 12:301-306 (1992)), and chemical mismatch cleavage (CMC) (Grompe et al., Proc. Natl. Acad. Sci. USA 86:5855-5892 (1989)). A review of currently available methods of detecting DNA sequence variation can be found in Grompe, Nature Genetics 5:111-117 (1993).

Seven well-known nucleic acid-based methods for confirming the presence of a polymorphism are described below. Provided for exemplary purposes only and not intended to limit any aspect of the invention, these methods include:

(1) single-stranded confirmation analysis (SSCA) (Orita et al.);

(2) denaturing gradient gel electrophoresis (DGGE) (Wartell et al., Nucl. Acids Res. 18:2699-2705 (1990) and Sheffield et al., Proc. Natl. Acad. Sci. USA 86:232-236 (1989));

(3) RNase protection assays (Finkelstein et al., Genomics 7:167-172 (1990) and Kinszler et al., Science 251:1366-1370 (1991));

(4) the use of proteins which recognize nucleotide mismatches, such as the E. Coli mutS protein (Modrich, Ann. Rev. Genet. 25:229-253 (1991);

(5) allele-specific PCR (Rano and Kidd, Nucl. Acids Res. 17:8392 (1989),), which involves the use of primers that hybridize at their 3′ ends to a polymorphism and, if the polymorphism is not present, an amplification product is not observed; and

(6) Amplification Refractory Mutation System (ARMS), as disclosed in European Patent Application Publication No. 0332435 and in Newton et al., Nucl. Acids Res. 17:2503-2516 (1989); and

(7) temporal temperature gradient gel electrophoresis (TTGE), as described by Bio-Rad in U.S./E.G. Bulletin 2103.

In SSCA, DGGE, TTGE, and RNase protection assay, a new electrophoretic band appears when the polymorphism is present. SSCA and TTGE detect a band that migrates differentially because the sequence change causes a difference in single-strand, intramolecular base pairing, which is detectable electrophoretically. RNase protection involves cleavage of the mutant polynucleotide into two or more smaller fragments. DGGE detects differences in migration rates of sequences compared to less pathogenic strain gnd sequences, using a denaturing gradient gel. In an allele-specific oligonucleotide assay (ASOs) (Conner et al., Proc. Natl. Acad. Sci. USA 80:278-282 (1983)), an oligonucleotide is designed that detects a specific sequence, and an assay is performed by detecting the presence or absence of a hybridization signal. In the mutS assay, the protein binds only to sequences that contain a nucleotide mismatch in a heteroduplex between polymorphic and non-polymorphic sequences. Mismatches, in this sense of the word refers to hybridized nucleic acid duplexes in which the two strands are not 100% complementary. The lack of total homology results from the presence of one or more polymorphisms in an amplicon obtained from a biological sample, for example, that has been hybridized to a non-polymorphic strand. Mismatched detection can be used to detect point mutations at Tyr719 or Tyr 721 in DNA or in its mRNA product. While these techniques are less sensitive than sequencing, they are easily performed on a large number of biological samples and are amenable to array technology.

In some embodiments, nucleic acid probes that differentiate polynucleotides encoding wild type Kit from mutant Kit are attached to a support in an ordered array, wherein the nucleic acid probes are attached to distinct regions of the support that do not overlap with each other. Preferably, such an ordered array is designed to be “addressable” where the distinct locations of the probe are recorded and can be accessed as part of an assay procedure. These probes are joined to a support in different known locations. The knowledge of the precise location of each nucleic acid probe makes these “addressable” arrays particularly useful in binding assays. The nucleic acids from a preparation of several biological samples are then labeled by conventional approaches (e.g., radioactivity or fluorescence) and the labeled samples are applied to the array under conditions that permit hybridization.

If a nucleic acid in the samples hybridizes to a probe on the array, then a signal will be detected at a position on the support that corresponds to the location of the hybrid. Since the identity of each labeled sample is known and the region of the support on which the labeled sample was applied is known, an identification of the presence of the polymorphic variant can be rapidly determined. These approaches are easily automated using technology known to those of skill in the art of high throughput diagnostic or detection analysis.

Additionally, an opposite approach to that presented above can be employed. Nucleic acids present in biological samples can be disposed on a support so as to create an addressable array. Preferably, the samples are disposed on the support at known positions that do not overlap. The presence of nucleic acids having a desired polymorphism in each sample is determined by applying labeled nucleic acid probes that complement nucleic acids that encode the polymorphism and detecting the presence of a signal at locations on the array that correspond to the positions at which the biological samples were disposed. Because the identity of the biological sample and its position on the array is known, the identification of the polymorphic variant can be rapidly determined. These approaches are also easily automated using technology known to those of skill in the art of high throughput diagnostic analysis.

Any addressable array technology known in the art can be employed with this aspect of the invention. One particular embodiment of polynucleotide arrays is known as Genechips™, and has been generally described in U.S. Pat. No. 5,143,854; PCT publications WO 90/15070 and 92/10092. These arrays are generally produced using mechanical synthesis methods or light directed synthesis methods, which incorporate a combination of photolithographic methods and solid phase oligonucleotide synthesis. (Fodor et al., Science, 251:767-777, (1991)). The immobilization of arrays of oligonucleotides on solid supports has been rendered possible by the development of a technology generally identified as “Very Large Scale Immobilized Polymer Synthesis” (VLSPIS™) in which, typically, probes are immobilized in a high density array on a solid surface of a chip. Examples of VLSPIS™ technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and diagnostic information. Examples of such presentation strategies are disclosed in PCT Publications WO 94/12305, WO 94/11530, WO 97/29212, and WO 97/31256.

A wide variety of labels and conjugation techniques are known by those skilled in the art and can be used in various nucleic acid assays. There are several ways to produce labeled nucleic acids for hybridization or PCR including, but not limited to, oligolabeling, nick translation, end-labeling, or PCR amplification using a labeled nucleotide. Alternatively, a nucleic acid encoding murine or human Kit, preferably residues 700-735, can be cloned into a vector for the production of an mRNA probe. Such vectors are known in the art, are commercially available, and can be used to synthesize RNA probes in vitro by addition of an appropriate RNA polymerase such as T7, T3 or SP6 and labeled nucleotides. A number of companies such as Pharmacia Biotech (Piscataway N.J.), Promega (Madison Wis.), and U.S. Biochemical Corp (Cleveland Ohio) supply commercial kits and protocols for these procedures. Suitable reporter molecules or labels include those radionuclides, enzymes, fluorescent, chemiluminescent, or chromogenic agents, as well as, substrates, cofactors, inhibitors, magnetic particles and the like.

The RNase protection method, briefly described above, is an example of a mismatch cleavage technique that is amenable to array technology. Preferably, the method involves the use of a labeled riboprobe that is complementary to a Kit receptor sequence having a polymorphism (such as the Y719X or Y721X polymorphism). However, the method can involve the use of a labeled riboprobe that is complementary to a Kit receptor sequence having the wild type gene. The riboprobe and either mRNA or DNA isolated and amplified from a biological sample are annealed (hybridized) and subsequently digested with the enzyme RNase A, which is able to detect mismatches in a duplex RNase structure. If a mismatch is detected by RNase A, the polymorphic variant is not present in the sample and the enzyme cleaves at the site of the mismatch and destroys the riboprobe. Thus, when the annealed RNA is separated on a electrophoretic gel matrix, if a mismatch has been detected and cleaved by RNase A, an RNA product will be seen which is much smaller than the full length duplex RNA for the riboprobe and the mRNA or DNA.

Complements to the riboprobe can also be dispersed on an array and stringently probed with the products from the Rnase A digestion after denaturing any remaining hybrids. In this case, if a mismatch is detected and probe destroyed by Rnase A, the complements on the array will not anneal with the degraded RNA under stringent conditions. In a similar fashion, DNA probes can be used to detect mismatches, through enzymatic or chemical cleavage. See, e.g., Cotton, et al., Proc. Natl. Acad. Sci. USA 85:4397 (1988); Shenk et al., Proc. Natl. Acad. Sci. USA 72:989 (1975); and Novack et al., Proc. Natl. Acad. Sci. USA 83:586 (1986). Mismatches can also be detected by shifts in the electrophoretic ability of mismatched duplexes relative to matched duplexes. (See, e.g., Cariello, Human Genetics 42:726 (1988),).

With any of the techniques described above, the mRNA or DNA from a tested subject that corresponds to regions of kit containing the Y719X or Y721X polymorphisms can be amplified by PCR before hybridization. A variety of PCR techniques are familiar to those skilled in the art. For a review of PCR technology, see Molecular Cloning to Genetic Engineering White, B. A. Ed. in Methods in Molecular Biology 67: Humana Press, Totowa (1997), and the publication entitled “PCR Methods and Applications” (1991, Cold Spring Harbor Laboratory Press). For amplification of mRNAs, it is within the scope of the present invention to reverse transcribe mRNA into cDNA followed by PCR (RT-PCR); or, to use a single enzyme for both steps as described in U.S. Pat. No. 5,322,770, or, to use Reverse Transcriptase Asymmetric Gap Ligase Chain Reaction (RT-AGLCR), as described by Marshall R. L. et al. (PCR Methods and Applications 4:80-84, 1994).

In each of these amplification procedures, primers on either side of the sequence to be amplified are added to a suitably prepared nucleic acid sample along with dNTPs and a thermostable polymerase such as Taq polymerase, Pfu polymerase, or Vent polymerase. The nucleic acid in the sample is denatured and the primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188.

The primers are selected to be substantially complementary to a portion of the sequence of Kit receptor DNA or mRNA and a portion of the sequence that complements the sequence of Kit receptor DNA or mRNA, thereby allowing the sequences between the primers to be amplified. Preferably, primers are 16, 17, 18, 19, 20, 21, 22, 23, 24, 25, 26, 27, 28, 29 and 30 nucleotides in length. Shorter primers tend to lack specificity for a target nucleic acid sequence and generally require cooler temperatures to form sufficiently stable hybrid complexes with the template. Longer primers are expensive to produce and can sometimes self-hybridize to form hairpin structures. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions may be empirically determined by one of skill in the art.

The spacing of the primers determines the length of the segment to be amplified. In the context of the present invention amplified segments carrying nucleic acid sequence encoding fragments of Kit can range in size from at least about 25 bp to 35 kb. Amplification fragments from 25-1000 bp are typical, fragments from 50-1000 bp are preferred and fragments from 100-600 bp are highly preferred. It will be appreciated that amplification primers can be of any sequence that allows for specific amplification of a region of the kit genes disclosed in SEQ ID NOs: 1 and 3 and can, for example, include modifications such as restriction sites to facilitate cloning.

The presence of a Kit receptor polymorphism or wild type sequence in a protein sample can also be detected by using conventional assays. For example, antibodies immunoreactive with Kit polymorphism (Y719X or Y721X) can be used to screen biological samples for the presence of a Kit-mediated fertility defect. Additionally, antibodies that differentiate the wild type receptor from mutant receptors (i.e., antibodies that recognize Y719 or Y721 receptors as opposed to Y719X or Y721X receptors) can be used to determine that a male does not have a Kit-mediated fertility defect. In preferred embodiments, antibodies are used to immunoprecipitate the wildtype or Y719X or Y721X mutant forms of the Kit receptor from solution or are used to react with the wild type or Y719X or Y721X forms of the Kit receptor on Western or Immunoblots. Favored diagnostic embodiments also include enzyme-linked immunosorbant assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530,. Other embodiments employ aspects of the immune-strip technology disclosed in U.S. Pat. Nos. 5,290,678; 5,604,105; 5,710,008; 5,744,358; and 5,747,274.

In another preferred protein-based diagnostic, antibodies of the present invention are attached to a support in an ordered array wherein a plurality of antibodies are attached to distinct regions of the support that do not overlap with each other. As with the nucleic acid-based arrays, the protein-based arrays are ordered arrays that are designed to be “addressable” such that the distinct locations are recorded and can be accessed as part of an assay procedure. These probes are joined to a support in different known locations. The knowledge of the precise location of each probe makes these “addressable” arrays particularly useful in binding assays. For example, an addressable array can comprise a support having several regions to which are joined a plurality of antibody probes that specifically recognize the Kit receptor and differentiate the murine and human wild type Kit receptors.

Proteins are obtained from biological samples and are labeled by conventional approaches (e.g., radioactivity, colorimetrically, or fluorescently). The labeled samples are then applied to the array under conditions that permit binding. If a protein in the sample binds to an antibody probe on the array, then a signal will be detected at a position on the support that corresponds to the location of the antibody-protein complex. Since the identity of each labeled sample is known and the region of the support on which the labeled sample was applied is known, an identification of the presence, concentration, and/or expression level can be rapidly determined. That is, by employing labeled standards of a known concentration of mutant Kit receptor, an investigator can accurately determine the protein concentration of Kit receptor in a tested sample and from this information can assess the expression level of the Kit receptor. Conventional methods in densitometry can also be used to more accurately determine the concentration or expression level of Kit receptor. These approaches are easily automated using technology known to those of skill in the art of high throughput diagnostic analysis.

In another embodiment, an opposite approach to that presented above can be employed. Proteins present in biological samples can be disposed on a support so as to create an addressable array. Preferably, the protein samples are disposed on the support at known positions that do not overlap. The presence of a protein encoding a specific form of mutant Kit receptor in each sample is then determined by applying labeled antibody probes that recognize epitopes of the Kit receptor that differentiate the Kit receptor from murine and human wild type receptors. Because the identity of the biological sample and its position on the array is known, an identification of the presence, concentration, and/or expression level of a particular polymorphism can be rapidly determined.

That is, by employing labeled standards of a known concentration of Kit receptor, an investigator can accurately determine the concentration of Kit receptor in a sample and from this information can assess the expression level of the Kit receptor. Conventional methods in densitometry can also be used to more accurately determine the concentration or expression level of the Kit receptor. These approaches are also easily automated using technology known to those of skill in the art of high throughput diagnostic analysis. As detailed above, any addressable array technology known in the art can be employed with this aspect of the invention and display the protein arrays on the chips in an attempt to maximize antibody binding patterns and diagnostic information.

As discussed above, the presence or detection of a polymorphism in a male subject can provide a diagnosis of a Kit-mediated fertility defect in the subject. Additional embodiments include the preparation of diagnostic kits comprising detection components, such as antibodies, specific for the Y721X Kit receptor. The detection component will typically be supplied in combination with one or more of the following reagents. A support capable of absorbing or otherwise binding RNA or protein will often be supplied. Available supports for this purpose include, but are not limited to, membranes of nitrocellulose, nylon or derivatized nylon that can be characterized by bearing an array of positively charged substituents, and Genechips™ or their equivalents. One or more enzymes, such as Reverse Transcriptase and/or Taq polymerase, can be furnished in the kit, as can dNTPs, buffers, or non-human polynucleotides like calf-thymus or salmon-sperm DNA. Results from the kit assays can be interpreted by a healthcare provider or a diagnostic laboratory. Alternatively, diagnostic kits are manufactured and sold to private individuals for self-diagnosis.

In the section below, several approaches to identify molecules that modulate the association of the intracellular domain of wild type and mutant Kit receptors with PI 3′-kinase are described. These modulators (i.e., agents that either inhibit or enhance the association of PI 3′-kinase with wild type or mutant Kit receptors) can be used in pharmaceuticals to inhibit or enhance spermatogenesis.

Approaches to Identify Agents That Modulate Spermatogenesis

The term “kinase activation assay”, the results of which can be recorded as a value in a “kinase activation profile”, include assays that directly or indirectly evaluate the activation state of a kinase molecule, such as PI 3′-kinase or Akt kinase, in the presence of a Kit receptor or fragment thereof. Additionally, these assays can be performed in the presence or absence of a Kit modulator (e.g., a “Kit inhibiting agent” or “Kit enhancing agent”). That is, kinase activation assays can include, but are not limited to, approaches to detect changes in phosphorylation of one or more proteins and assays that evaluate the binding of one or more proteins in the presence and/or absence of a Kit modulator.

In some kinase activation assays, for example, the intact wild type murine or human Kit receptor or peptides comprising regions of the intracellular domain of the murine or human Kit receptor are phosphorylated and binding to PI 3′kinase is determined in the presence and absence of a Kit modulator (i.e., an agent that either inhibits or enhances Kit-mediated activation of PI 3′-kinase). In other kinase activation assays, the Kit receptors are contacted with a Kit enhancing agent (i.e., an agent that enhances Kit-mediated activation of PI 3′-kinase as opposed to a “Kit inhibiting agent”, which inhibits Kit-mediated activation of PI 3′-kinase) in the presence of ATP and binding to PI 3′-kinase is determined. Additionally, Kinase activation assays can include cell based assays in which the mutant or wild type Kit receptors are expressed in cells, the cells are contacted with a Kit inhibiting or enhancing agent and Kit receptor associated PI 3′-kinase activation is determined.

Some of the methods described above involve binding assays that utilize multimeric agents. One form of multimeric agent concerns a manufacture comprising murine or human (wild type or mutant) Kit receptors or fragments of these receptors having regions of their intracellular domains, disposed on a support. These multimeric agents provide the Kit receptors or fragments thereof in such a form or in such a way that a sufficient affinity for PI 3′-kinase can be achieved. A multimeric agent having murine or human (wild type or mutant) Kit receptors or fragments thereof is obtained by joining the desired polypeptide to a macromolecular support. A “support” can be a termed a carrier, a protein, a resin, a cell membrane, or any macromolecular structure used to join or immobilize such molecules. Solid supports include, but are not limited to, the walls of wells of a reaction tray, test tubes, polystyrene beads, magnetic beads, nitrocellulose strips, membranes, microparticles such as latex particles, animal cells, Duracyte®, artificial cells, and others. Kit receptors or fragments thereof can also be joined to inorganic carriers, such as silicon oxide material (e.g. silica gel, zeolite, diatomaceous earth or aminated glass) by, for example, a covalent linkage through a hydroxy, carboxy or amino group and a reactive group on the carrier.

In several mutimeric agents, the macromolecular support has a hydrophobic surface that interacts with a portion of the Kit receptor or fragment thereof by a hydrophobic non-covalent interaction. In some cases, the hydrophobic surface of the support is a polymer such as plastic or any other polymer in which hydrophobic groups have been linked such as polystyrene, polyethylene or polyvinyl. Additionally, Kit receptors or fragments thereof can be covalently bound to carriers including proteins and oligo/polysaccarides (e.g. cellulose, starch, glycogen, chitosane or aminated sepharose). In these later multimeric agents, a reactive group on a molecule, such as a hydroxy or an amino group, is used to join to a reactive group on the carrier so as to create the covalent bond. Additional multimeric agents comprise a support that has other reactive groups that are chemically activated so as to attach the Kit receptor or fragment thereof. For example, cyanogen bromide activated matrices, epoxy activated matrices, thio and thiopropyl gels, nitrophenyl chloroformate and N-hydroxy succinimide chlorformate linkages, or oxirane acrylic supports are used. (Sigma).

By using a cell based approach, many candidate Kit modulators can be rapidly screened for their ability to inhibit or enhance association or activation of PI 3′-kinase with a Kit receptor and/or the ability to activate Akt and, thereby, identify agents that modulate spermatogenesis. For example, Chinese hamster ovary cells or NIH 3T3 cells can be transfected with a construct that will express a wild type or mutant Kit receptor. The overexpression of Kit in these cell lines can be performed as known in the art. (See Levy et al., Proc. Natl. Acad. Sci. USA 89: 678-682 (1992).) Expressing cells can then be contacted with molecules obtained from peptidomimetic and/or combinatorial chemistry libraries. After incubation with the candidate Kit modulators, the effect on PI 3′-kinase binding to the Kit receptor and/or the ability to activate Akt can be evaluated. By comparing the level of PI 3′-kinase binding and/or Akt activation in control cells, which were not contacted with the candidate Kit modulator, to cells incubated with the various candidate modulators, molecules that inhibit or enhance spermatogeneisis can be identified.

Another approach to identify agents that inhibit spermatogenesis, employs wild type Kit receptor or a fragment thereof having the intracellular domain of the receptor provided on a resin. (See Levy et al., Proc. Nat. Acad. Sci. USA 89: 678-682 (1992).) Accordingly, the Kit receptor or fragment thereof is phosphorylated, for example by autophosphorylation or by incubation with immunoprecipitates of a receptor protein tyrosine kinase (e.g., Kit itself in the presence of [γ-³²P] ATP or the EGF receptor isolated from A431 cells). The phosphorylated Kit receptor or Kit fragment on the support is then contacted with a candidate Kit inhibiting agent and PI 3′-kinase, under conditions that allow for binding. Controls for the experiment are run in which the PI 3′-kinase is allowed to bind to the Kit-containing multimeric agent in the absence of the Kit inhibiting agent. By comparing the amount of PI 3′ binding and/or activation in the controls to the assays having the candidate Kit inhibiting agent, one can rapidly identify molecules that inhibit Kit-mediated activation of PI 3′-kinase. Candidate molecules for such an assay include peptides, peptidomimetics, and chemicals that resemble regions of the intracellular domain of the murine or human Kit receptor (e.g., corresponding to amino acid position 700-735 of Kit) and peptides, peptidomimetics, and chemicals that bind to regions of the intracellular domain of the murine or human Kit receptor (e.g., amino acid position 700-735 of Kit) and thereby modulate Kit-induced PI 3′-kinase activation

In a similar manner, agents that enhance spermatogenesis can be identified. Accordingly, wild type or mutant Kit receptor (human or murine) or a fragment thereof having the intracellular domain of the receptor are provided on a multimeric agent. Next, the Kit receptor or fragment thereof is phosphorylated, as described above. The Kit receptor or Kit fragment on the support is then contacted with a candidate Kit enhancing agent and PI 3′-kinase, under conditions that allow for binding. Controls for the experiment are run in which the PI 3′-kinase is allowed to bind to the Kit-containing multimeric agent in the absence of the Kit enhancing agent. By comparing the amount of PI 3′ binding and/or activation of PI 3′-kinase in the controls to the assays having the candidate Kit enhancing agent, one can rapidly identify molecules that enhance Kit-mediated activation of PI 3′-kinase.

Other approaches to identify Kit modulators take advantage of transcription assays that analyze protein:protein interactions. That is, several methods or procedures that identify compounds that modulate transcription by enhancing or inhibiting protein:protein interactions can be adapted to identify Kit modulators by using the methodology described above. For example, the following assays can be adapted to identify kit modulators:

(1) the two-hybrid systems (Field & Song, Nature 340:245-246 (1989); Chien et al., Proc. Natl. Acad. Sci. USA 88:9578-9582 (1991); and Young K H, Biol. Reprod. 58:302-311 (1998));

(2) reverse two-hybrid system (Leanna & Hannink, Nucl. Acid Res. 24:3341-3347 (1996));

(3) repressed transactivator system (Sadowski et al., U.S. Pat. No. 5,885,779));

(4) phage display (Lowman H B, Annu. Rev. Biophys. Biomol. Struct. 26:401-424 (1997)); and

(5) GST/HIS pull down assays, mutant operators (Granger et al., WO 98/01879) and the like (See also Mathis G., Clin. Chem. 41:139-147 (1995); Lam K. S. Anticancer Drug Res., 12:145-167 (1997); and Phizicky et al., Microbiol. Rev. 59:94-123 (1995)).

In this manner, “kinase activation profiles” comprising the values and results (e.g., binding or activation of PI 3′ kinase or levels of Akt activation) from one or more kinase activation assays can be generated. These kinase activation profiles can then be compiled to create “a Kit modulator profile”. A Kit modulator profile can be composed of a chemical structure, nucleic acid sequence, or polypeptide sequence or model of an agent that modulates (inhibits or enhances) Kit-mediated activation of PI 3′-kinase, one or more symbols that represent these molecules and/or models, an identifier that represents a class of such agents, and a value or result from a kinase activation assay. By comparing Kit modulator profiles, derivative Kit modulators can be rationally selected and created by further rounds of combinatorial chemistry, as described in the following section.

Identification of Kit Modulators by Rational Drug Design

One goal of rational drug design is to produce structural analogs of biologically active polypeptides of interest or of small molecules with which they interact (e.g., agonists, antagonists, null compounds) in order to fashion drugs that are, for example, more or less potent forms of the molecule. (See, e.g., Hodgson, Bio. Technology 9:19-21 (1991)). Rational drug design has been used to develop HIV protease inhibitors and agonists for five different somatostatin receptor subtypes. (Erickson et al., Science 249:527-533 (1990) and Berk et al., Science 282:737 (1998)).

Given the nucleic acid or protein sequence of a Kit modulator, for example, stable analogs of these molecules or portions thereof can be created. These small molecules are referred to as peptidomimetics. A peptidomimetic is a molecule that has the same effect as a peptide, usually because it has the same critical ‘shape’, but is not itself a peptide and hence is not broken down by proteases and is less expensive to produce. Thus, peptidomimetics that structurally and/or functionally resemble Kit modulators can be made and evaluated for their ability to modulate Kit-mediated activation of PI 3′-kinase. Several approaches to make peptidomimetics that resemble polypeptides are described in the art. A number of methods, for example, can be found in U.S. Pat. Nos. 5,288,707; 5,552,534; 5,811,515; 5,817,626; 5,817,879; 5,821,231; and 5,874,529.

Rational drug design is also preferably performed with the aid of computer technology. For example, the murine and/or human, protein sequence of wild type and mutant Kit receptors, or the intracellular domains of these receptors (or nucleic acid sequence encoding these polypeptides or both), can be entered onto a computer readable medium for recording and manipulation. It will be appreciated by those skilled in the art that a computer readable medium having these sequences can interface with software that converts or manipulates the sequences to obtain structural and functional information, such as protein models. That is, the functionality of a software program that converts or manipulates these sequences includes the ability to compare these sequences to other sequences or structures of molecules that are present on publicly and commercially available databases so as to conduct rational drug design.

The wild type or mutant Kit protein or fragments of Kit including the intracellular domain of the receptor, as well as, nucleic acids encoding these polypeptides or both can be stored, recorded, and manipulated on any medium that can be read and accessed by a computer. As used herein, the words “recorded” and “stored” refer to a process for storing information on computer readable medium. A skilled artisan can readily adopt any of the presently known methods for recording information on a computer readable medium to generate manufactures comprising the nucleotide or polypeptide sequence information of this embodiment. A variety of data storage structures are available to a skilled artisan for creating a computer readable medium having recorded thereon a nucleotide or polypeptide sequence. The choice of the data storage structure will generally be based on the component chosen to access the stored information. Computer readable media include magnetically readable media, optically readable media, or electronically readable media. For example, the computer readable media can be a hard disc, a floppy disc, a magnetic tape, zip disk, CD-ROM, DVD-ROM, RAM, or ROM as well as other types of other media known to those skilled in the art. The computer readable media on which the sequence information is stored can be in a personal computer, a network, a server or other computer systems known to those skilled in the art.

Embodiments of the invention utilize computer-based systems that contain the sequence information described herein and convert this information into other types of usable information (e.g., protein models for rational drug design). The term “a computer-based system” refers to the hardware, software, and any database used to analyze the wild type or mutant kit nucleic acid sequence or the wild type or mutant Kit protein sequence or both, or fragments of these biomolecules (particularly fragments that correspond to the intracellular domain of the receptor) so as to construct models or to conduct rational drug design. The computer-based system preferably includes the storage media described above, and a processor for accessing and manipulating the sequence data. The hardware of the computer-based systems of this embodiment comprise a central processing unit (CPU) and a database. A skilled artisan can readily appreciate that any one of the currently available computer-based systems are suitable.

In one particular embodiment, the computer system includes a processor connected to a bus that is connected to a main memory (preferably implemented as RAM) and a variety of secondary storage devices, such as a hard drive and removable medium storage device. The removable medium storage device may represent, for example, a floppy disk drive, a DVD drive, an optical disk drive, a compact disk drive, a magnetic tape drive, etc. A removable storage medium, such as a floppy disk, a compact disk, a magnetic tape, etc. containing control logic and/or data recorded therein can be inserted into the removable storage device. The computer system includes appropriate software for reading the control logic and/or the data from the removable medium storage device once inserted in the removable medium storage device. The murine and/or human, wild type or mutant kit nucleic acid sequence or the murine and/or human, wild type or mutant Kit protein sequence or both can be stored in a well known manner in the main memory, any of the secondary storage devices, and/or a removable storage medium. Software for accessing and processing these sequences (such as search tools, compare tools, and modeling tools etc.) reside in main memory during execution.

As used herein, “a database” refers to memory that can store kit nucleotide or Kit polypeptide sequence information, protein model information, information on other peptides, chemicals, peptidomimetics, and other agents that interact with Kit proteins, and values or results from kinase activation assays. Additionally, a “database” refers to a memory access component that can access manufactures having recorded thereon kit nucleotide or Kit polypeptide sequence information, protein model information, information on other peptides, chemicals, peptidomimetics, and other agents that interact with Kit proteins, and values or results from kinase activation assays. In other embodiments, a database stores a “kinase activation profile” comprising the values and results (e.g., levels of PI 3′-kinase and/or Akt activation) from one or more “kinase activation assays”, as described herein or known in the art, and relationships between these values or results. The sequence data and values or results from kinase activation assays can be stored and manipulated in a variety of data processor programs in a variety of formats. For example, the sequence data can be stored as text in a word processing file, such as Microsoft WORD or WORDPERFECT, an ASCII file, a html file, or a pdf file in a variety of database programs familiar to those of skill in the art, such as DB2, SYBASE, or ORACLE.

A “search program” refers to one or more programs that are implemented on the computer-based system to compare a kit nucleotide or Kit polypeptide sequence with other nucleotide or polypeptide sequences and agents including but not limited to peptides, peptidomimetics, and chemicals stored within a database. A search program also refers to one or more programs that compare one or more protein models to several protein models that exist in a database and one or more protein models to several peptides, peptidomimetics, and chemicals that exist in a database. A search program is used, for example, to compare one kinase activation profile to one or more kinase activation profiles that are present in a database. Still further, a search program can be used to compare values or results from kinase activation assays and agents that modulate Kit-mediated activation of PI 3′-kinase.

A “retrieval program” refers to one or more programs that can be implemented on the computer-based system to identify a homologous nucleic acid sequence, a homologous protein sequence, or a homologous protein model. A retrieval program can also used to identify peptides, peptidomimetics, and chemicals that interact with a Kit protein sequence, or a Kit protein model stored in a database. Further, a retrieval program is used to identify a specific agent that modulates Kit-mediated activation of PI 3′-kinase to a desired set of values, results, or kinase activation profile. That is, a retrieval program can also be used to obtain “a Kit modulator profile” that is composed of a chemical structure, nucleic acid sequence, or polypeptide sequence or model of an agent that modulates (inhibits or enhances) Kit-mediated activation of PI 3′-kinase, one or more symbols that represent these molecules and/or models, an identifier that represents one or more agents including, but not limited to peptides and peptidomimetics (referred to collectively as “peptide agents”) and chemicals, and a value or result from a kinase activation assay.

In some rational drug design approaches two dimensional and three dimensional models of wild type or mutant murine or human Kit receptor sequences or both are created and regions of the intracellular domain of these receptors that are involved in Kit-mediated activation of PI 3′-kinase are analyzed. These models can be used by one of skill to rationally design molecules that modulate Kit induced PI 3′-kinase activation (“Kit modulators”), such as peptides, peptidomimetics, and chemicals. In some embodiments, Kit receptor sequences or models thereof are created and analyzed so that molecules that block kinase activation (“Kit inhibiting agents”), such as peptides, peptidomimetics, and chemicals, can be predicted and designed. In other embodiments, mutant Kit receptor sequences or models thereof are created and analyzed so that molecules that circumvent this block on kinase activation (“Kit enhancing agents”), such as peptides, peptidomimetics, and chemicals, can be predicted and designed. Molecules identified by these computer aided approaches are preferably verified in kinase activation assays and kinase activation and Kit modulator profiles are generated.

A protein model of the intact murine or human Kit receptor or regions of the intracellular domain of the Kit receptor is a starting point for computer aided rational drug design. Perhaps the best known way of determining protein structure involves the use of x-ray crystallography. A general review of this technique can be found in Van Holde, K. E. Physical Biochemistry, Prentice-Hall, N.J. pp. 221-239 (1971).

Investigators have solved the crystal structure of the amino terminal SH2 domain of the p85 α subunit of PI 3′-kinase, alone and in complex with phosphopeptides bearing the consensus binding motif. (Nolte et al., Nature Struct. Biol., 3:364-373 (1996)). These crystal structures can be used to develop molecules that inhibit the interaction of p85 with PI 3′-kinase. A more complete understanding of the interaction of p85 with the Kit receptor can be obtained, however, from crystal structures of p85 with larger fragments of the Kit receptor, preferably a fragment corresponding to amino acid residues 687-763 or 700-735.

To obtain models of p85 with larger fragments of the Kit receptor, techniques such as neutron diffraction, or by nuclear magnetic resonance (NMR) can be used in addition to x-ray crystallography. (See, e.g., Moore, W. J., Physical Chemistry, 4^{th} Edition, Prentice-Hall, N.J. (1972)). Three dimensional protein models of p85 with larger fragments of the Kit receptor can also be constructed using computer-based protein modeling techniques. By one approach, the protein folding problem is solved by finding target sequences that are most compatible with profiles representing the structural environments of the residues in known three-dimensional protein structures. (See, e.g., Eisenberg et al., U.S. Pat. No. 5,436,850 issued Jul. 25, 1995). In another technique, the known three-dimensional structures of proteins in a given family are superimposed to define the structurally conserved regions in that family. This protein modeling technique also uses the known three-dimensional structure of a homologous protein to approximate the structure of a polypeptide of interest. (See e.g., Srinivasan, et al., U.S. Pat. No. 5,557,535 issued Sep. 17, 1996). Conventional homology modeling techniques have been used routinely to build models of proteases and antibodies. (Sowdhamini et al., Protein Engineering 10:207, 215 (1997)). Comparative approaches can also be used to develop three-dimensional protein models when the protein of interest has poor sequence identity to template proteins.

In some cases, proteins fold into similar three-dimensional structures despite having very weak sequence identities. For example, the three-dimensional structures of a number of helical cytokines fold in similar three-dimensional topology in spite of weak sequence homology. The recent development of threading methods and “fuzzy” approaches now enables the identification of likely folding patterns and functional protein domains in a number of situations where the structural relatedness between target and template(s) is not detectable at the sequence level. By one method, fold recognition is performed using Multiple Sequence Threading (MST) and structural equivalences are deduced from the threading output using the distance geometry program DRAGON that constructs a low resolution model. A full-atom representation is then constructed using a molecular modeling package such as QUANTA. According to this 3-step approach, candidate templates are first identified by using the novel fold recognition algorithm MST, which is capable of performing simultaneous threading of multiple aligned sequences onto one or more 3-D structures.

In a second step, the structural equivalences obtained from the MST output are converted into inter-residue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Aszódi et al., Proteins: Structure, Function, and Genetics, Supplement 1:38-42 (1997)).

After obtaining a model of p85 bound to the intracellular region of the Kit receptor or fragment thereof, libraries of molecules that resemble various regions of the intracellular domain of the Kit receptor can be rapidly designed, created, tested in kinase activation assays, and used as templates to design more Kit modulators. For example, the crystal structure of the amino terminal SH2 domain of the p85 a subunit of PI 3′-kinase, alone and in complex with phosphopeptides bearing a consensus binding motif as described by Nolte et al., Nature Struct Biol., 3:364-373 (1996), can be used to design and create candidate Kit modulators.

One contemplated agent is a peptide having the amino acid sequence TNEpYMDMKPEV (SEQ ID NO: 5). Another contemplated agent is a peptide having the formula pYVPMLDMK (SEQ ID NO: 6). Other contemplated Kit inhibitors have the general amino acid formula X¹ₙpYMX²X³MX⁴ₙ, wherein: “X¹ₙ” denotes any amino acid residue in any order numbering between 1-1000 residues; “p” denotes phosphorylation; “Y” denotes tyrosine; “M” denotes methionine; X² denotes a member of the group consisting of methionine, valine, isoleucine, and glutamine; and “X⁴ₙ” denotes any amino acid residue in any order numbering between 1-1000 residues.

These candidate Kit modulators can be verified in kinase activation assays and molecules identified as inhibiting PI 3′-kinase activation can be used as templates to create other Kit modulators such as peptidomimetics and chemicals that resemble these molecules and derivatives thereof. Techniques in combinatorial chemistry are contemplated for use in derivatizing molecules that resemble the above-identified compounds.

In some contexts, the terms “Kit modulator” or “Kit inhibiting agent” or “Kit enhancing agent” includes polypeptide fragments corresponding to regions of the intracellular domain of murine or human Kit, fusion proteins comprising regions of the intracellular domain of murine or human Kit, nucleic acids encoding these molecules, and derivatized peptides, peptidomimetics and chemicals resembling these molecules. Similarly, libraries of molecules that bind to or associate with the intracellular domain of the murine or human Kit receptor and thereby modulate Kit-mediated activation of PI 3′-kinase can be created. That is, in some contexts, the terms “Kit modulator” or “Kit inhibiting agent” or “Kit enhancing agent” refers to molecules that bind to regions of the intracellular domain of the murine or human Kit receptor including, but not limited to, derivatized peptides, antibodies, peptidomimetics, and chemicals. In a similar manner to that described above, molecules (e.g., fragments of antibodies) that bind to the wild type or mutant intracellular region of the Kit receptor can be modeled and peptidomimetics, chemicals, and derivative molecules can be made from these templates. These candidate molecules are also preferably evaluated in kinase activation assays, profiles are obtained, and further cycles of rational drug design are conducted.

In another embodiment, computer modeling and the sequence-to-structure-to-function paradigm is exploited to identify more Kit modulating agents. By this approach, first the structure of a Kit modulator (e.g., an antibody that binds to epitopes on the Kit receptor presented by amino acid residues 687-763 and thereby inhibits Kit-mediated activation of PI 3′kinase) is determined from its sequence using a threading algorithm, which aligns the sequence to the best matching structure in a structural database. Next, the protein's active site (i.e., the site important for inhibiting kinase activation) is identified and a “fuzzy functional form” (FFF)—a three-dimensional descriptor of the active site of a protein—is created. (See e.g., Fetrow et al., J. Mol. Biol. 282:703-711 (1998) and Fetrow and Skolnick, J. Mol. Biol. 281: 949-968 (1998)). The active site of the Kit modulator can also be determined by using conventional mutagenesis techniques, for example, alanine scan. In alanine scan an amino acid residue of the agent is replaced by alanine, and its affect on the peptide's activity is measured by functional assays, such as the kinase activation assays described herein. Each of the amino acid residues of the peptide is analyzed in this manner and the regions important for inhibiting kinase activation are identified.

The FFFs are built by iteratively superimposing the protein geometries from a series of functionally related proteins with known structures. The FFFs are not overly specific, however, and the degree to which the descriptors can be relaxed is explored. In essence, conserved and functionally important residues for a desired response are identified and a set of geometric and conformational constraints for a specific function are defined in the form of a computer algorithm. The program then searches experimentally determined protein structures from a protein structural database for sets of residues that satisfy the specified constraints. In this manner, homologous three-dimensional structures can be compared and degrees (e.g., percentages of three-dimensional homology) can be ascertained.

By using this computational protocol, genome sequence data bases such as maintained by various organizations including: http://www.tigr.org/tdb; http://www.genetics.wisc.edu; http://genome-www.stanford.edu/˜ball: http://hiv-web.lanl.gov; http://wwwncbi.nlm.nih.gov; http://www.ebi.ac.uk; http://pasteur.fr/other/biology: and http://www-genome.wi.mit.edu, can be rapidly screened for specific protein active sites and for identification of the residues at those active sites that resemble a desired molecule. Several other groups have developed databases of short sequence patterns or motifs designed to identify a given function or activity of a protein. These databases, notably Prosite (http://expasy.hcuge.ch/sprot/prosite.html); Blocks (http://www.blocks.fhcrc.org); and Prints (http://www.biochem.ucl.ac.uk/bsm/dbbrowser/PRINTS/PRINTS.html), use short stretches of sequence information to identify sequence patterns that are specific for a given function; thus they avoid the problems arising from the necessity of matching entire sequences. In this manner, new Kit modulators are rationally selected for further identification. Additionally, the FFFs and homologous sequence information can be used to develop new Kit modulators using combinatorial chemistry. Classes of molecules that correspond to specific FFFs can then be tested in the kinase activation assays. Rounds or cycles of kinase activation assays on the molecules and derivatives thereof and further FFF refinement and database searching allows an investigator to more narrowly define classes of Kit modulators that produce a desired Kit-mediated activation of PI 3′-kinase.

Many computer programs and databases can be used with embodiments of the invention to identify agents that modulate Kit-mediated activation of PI 3′-kinase. The following list is intended not to limit the invention but to provide guidance to programs and databases that are useful with the approaches discussed above. The programs and databases that may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403 (1990)), FASTA (Pearson and Lipman, Proc. Natl. Acad. Sci. USA, 85: 2444 (1988)), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), Modeller 4 (Sali and Blundell J. Mol. Biol. 234:217-241 (1997)), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, and the BioByteMasterFile database. Many other programs and data bases would be apparent to one of skill in the art given the present disclosure. The following section describes the preparation of pharmaceuticals having Kit modulators, which can be administered to subjects in need to modulate spermatogenesis.

The Manufacture and Dose of Pharmaceuticals That Modulate Spermatogenesis

The Kit inhibiting agents identified by the methods of the invention are suitable for incorporation into pharmaceuticals that treat subjects in need as a preventive measure to inhibit spermatogenesis (e.g., for contraception or to preserve fertility by inhibiting germ cell proliferation during cancer chemotherapy). The Kit enhancing agents identified by the methods of the invention are suitable for incorporation into pharmaceuticals that treat subjects in need as a treatment measure to promote spermatogenesis (e.g., to enhance the fertility in males suffering from a Kit-mediated fertility defect). These pharmacologically active compounds can be processed in accordance with conventional methods of galenic pharmacy to produce medicinal agents for administration to subjects, e.g., mammals including humans. The active ingredients can be incorporated into a pharmaceutical product with and without modification. Further, the manufacture of pharmaceuticals or therapeutic agents that deliver the pharmacologically active compounds of this invention by several routes are aspects of the invention. For example, and not by way of limitation, DNA, RNA, and viral vectors having sequence encoding the Kit modulators are used with embodiments. Nucleic acids encoding Kit modulators can be administered alone or in combination with other active ingredients.

The compounds of this invention can be employed in admixture with conventional excipients, i.e., pharmaceutically acceptable organic or inorganic carrier substances suitable for parenteral, enteral (e.g., oral) or topical application that do not deleteriously react with the pharmacologically active ingredients of this invention. Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable vehicles are described in Remmington's Pharmaceutical Sciences, 15th Edition, Easton:Mack Publishing Company, pages 1405-1412 and 1461-1487(1975) and The National Formulary XIV, 14th Edition, Washington, American Pharmaceutical Association (1975). The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The effective dose and method of administration of a particular pharmaceutical formulation having a Kit modulators can vary based on the individual needs of the patient and the treatment or preventative measure sought. Therapeutic efficacy and toxicity of such compounds can be determined by standard pharmaceutical procedures in cell cultures or experimental animals, e.g., ED50 (the dose therapeutically effective in 50% of the population). For example, Kit enhancing agents identified by the in vitro screens discussed above, can be administered to Y719F mice and the effect on male fertility can be determined. The data obtained from these assays is then used in formulating a range of dosage for human use. The dosage of such compounds lies preferably within a range of circulating concentrations that include the ED50 with no toxicity. The dosage varies within this range depending upon type of Kit modulator, the dosage form employed, sensitivity of the patient, and the route of administration.

Normal dosage amounts of various Kit modulators may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, and 10 g.

In some embodiments, the dose of Kit modulators preferably produces a tissue or blood concentration or both from approximately 0.1 μM to 500 mM. Desirable doses produce a tissue or blood concentration or both of about 1 to 800 μM. Preferable doses produce a tissue or blood concentration of greater than about 10 μM to about 500 μM. Preferable doses are, for example, the amount of Kit modulators required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 145 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration of greater than 800 μM are not preferred, they can be used with some embodiments of the invention. A constant infusion of the Kit modulators can also be provided so as to maintain a stable concentration in the tissues as measured by blood levels.

The exact dosage is chosen by the individual physician in view of the patient to be treated. Dosage and administration are adjusted to provide sufficient levels of the active moiety or to maintain the desired effect. Additional factors that can be taken into account include the severity of the fertility defect, age of the patient, age, and weight of the patient; diet, time and frequency of administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once, twice, three, four, five, six, seven, eight, nine, ten or more times per day.

Routes of administration of the pharmaceuticals of the invention include, but are not limited to, transdermal, parenteral, gastrointestinal, transbronchial, and transalveolar. Transdermal administration is accomplished by application of a cream, rinse, gel, etc. capable of allowing the pharmacologically active compounds to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Compositions having the pharmacologically active compounds of this invention that are suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al.

Compositions having the pharmacologically active compounds of this invention that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the pharmacologically active compounds of this invention that are suitable for transbronchial and transalveolar administration include, but not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver compositions having the pharmacologically active compounds of the invention.

Compositions having the pharmacologically active compounds of this invention that are suitable for gastrointestinal administration include, but not limited to, pharmaceutically acceptable powders, pills or liquids for ingestion and suppositories for rectal administration. Due to the ease of use, gastrointestinal administration, particularly oral, is a preferred embodiment. Once the pharmaceutical comprising the Kit modulator has been obtained, it can be administered to a subject in need to treat Kit mediated infertility or prevent spermatogenesis. The section below describes several methods of modulating spermatogenesis so as to provide for male contraception and treat Kit-mediated fertility defects.

Therapeutic and Prophylactic Approaches

In several aspects of the invention, Kit modulators, in particular pharmaceuticals having Kit modulators, are provided to a subject in need to prevent spermatogenesis or treat Kit-mediated fertility defects. Methods to formulate pharmaceuticals that modulate (inhibit or enhance) Kit-mediated activation of PI 3′-kinase and, therefore, spermatogeneisis are embodiments of the invention. Further, embodiments of the invention include the use of medicaments comprising a Kit modulator for the inhibition or enhancement of Kit-mediated PI 3′-kinase activation and concomitantly the inhibition or enhancement of spermatogenesis.

In one embodiment, a Kit inhibiting agent can be used to inhibit spermatogenesis for the purposes of contraception. Accordingly, a male subject desiring contraception is provided a sufficient quantity of Kit inhibiting agent so that spermatogenesis is curtailed. Desirable Kit inhibiting agents for use in this method bind to or mimic regions of the intracellular domain of the Kit receptor. Preferable Kit inhibiting agents for use with this method bind to or mimic regions of the intracellular domain of the Kit receptor within amino acid residues 687-763. That is preferred Kit inhibiting agents bind to or mimic regions of the Kit receptor that can include amino acid residues 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762 and 763. Dosage of the Kit inhibiting agent is continued for the course in which contraception is desired.

In a similar aspect of the invention, a Kit inhibiting agent can be used to inhibit spermatogenesis for the purposes of preserving fertility by inhibiting germ cell proliferation during cancer chemotherapy. During chemotherapy, the rapidly dividing germ cells of a subject frequently die, effectively sterilizing the individual. To prevent the die off of rapidly diving germ cells in males about to under go such cancer therapy, the inventors have discovered a prophylactic method that utilizes a Kit inhibiting agent. Accordingly, prior to and during chemotherapy, a subject is provided a sufficient dose of Kit inhibiting agent to suppress spermatogenesis and, thus, protect the germ cells from the chemotherapeutic agent. Desirable Kit inhibiting agents for use in this method bind to or mimic regions of the intracellular domain of the Kit receptor. Preferable Kit inhibiting agents for use with this method bind to or mimic regions of the intracellular domain of the Kit receptor within amino acid residues 687-763. That is preferred Kit inhibiting agents bind to or mimic regions of the Kit receptor that can include amino acid residues 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762 and 763. Dosage of the Kit inhibiting agent is continued for the course in which the chemotherapy is conducted.

Still another aspect of the invention is directed to a method of treating a subject suffering from a Kit-mediated fertility defect. Accordingly, a subject suffering from a Kit-mediated fertility defect is first identified and then is administered a therapeutically effective amount of a Kit enhancing agent so that PI 3′ kinase can be associated with the Kit receptor and spermatogenesis can resume. Subjects suffering from Kit-mediated fertility defects can be identified by using the diagnostic approaches described in this disclosure. Desirable Kit enhancing agents for use in this method link regions of PI 3′ kinase to regions of the intracellular domain of the Kit receptor and thereby permit phosphorylation of PI 3′ kinase. Preferable Kit enhancing agents for use with this method link regions of PI 3′ kinase to regions of the intracellular domain of the Kit receptor within amino acid residues 687-763. That is preferred Kit enhancing agents link regions of PI 3′ kinase to regions of the intracellular domain of the Kit receptor that can include amino acid residues 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762 and 763. Dosage of the Kit enhancing agent is continued for the course in which the enhanced fertility is desired.

The following four examples describe a method that was used to make and analyze the Y719F mouse.

### EXAMPLE 1

### Gene Targeting, Generation of Prm-Cre Embryonic Stem Cells, and Derivation of Y719F-Kit/SCF-R Homozygous Mutant Mice

A 13.6-kb BamHI/BamHI genomic Kit fragment covering exon 15 was cloned from a 129/Sv mouse genomic library. To create a targeting construct, a 1.2-kb loxP-flanked neo cassette from pOG277 was cloned into the intron 606 bp upstream of exon 15. (FIG. 1(a)). A 1.5-kb Xba fragment covering exons 15 and 16 was subjected to site-directed mutagenesis, which changed the codon encoding Y719 to Y719F and, simultaneously, introduced a diagnostic EcoRI site (i.e., TAT was changed to TTC). The mutated fragment was blunted and, after complete sequence verification, used to replace the wild type 1.5-kb fragment (FIG. 1(a)). A unique NotI site 3′ of the homologous sequence was used to linearize the targeting construct before electroporation into 129/SvJae Prm-Cre ES cells. (O'Gorman et al., Proc. Natl. Acad. Sci. USA, 94:14602-7 (1997)).

Transfected Prm-Cre ES cell clones were selected in G418, and resistant colonies were screened by Southern blot. Internal and external probes (FIG. 1(a), and below) were used to verify correct targeting and absence of concatamers in the several clones that contained the Y719F mutation. A cross-over event between the neo cassette and the Y719F mutation was identified in one clone, which consequently had wild type sequence over Y719. This ES cell clone was introduced into the germ line of mice and served as a loxP-containing control. Three Y719F/+ and the control ES cell clones were injected into C57BL/6J blastocysts. Five chimeras derived from a Y719F/+ ES cell clone and two from the control ES clone, respectively, were transmitted through the germ line. The chimeric mice were crossed with B6D2F1/J mice and inbred with 129/Sv-+^{ρ}+^{tyr-c}+^{Mgf-SIJ}/J mice. The resulting mice were intercrossed to obtain homozygous mutant mice of both mixed (B6D2F1x129/Sv) or inbred (129/Sv) background.

### EXAMPLE 2

### Genotyping

Southern blotting and PCR on genomic DNA was performed on both ES cell and mouse DNA. To pre-screen for homologous recombination in the ES cells, genomic DNA from 192 G418-resistant ES cell clones was digested with XbaI. A Southern blot was performed using the 3′ external probe A (FIG. 1(a), which gave a targeting frequency of 21%. Genomic DNA samples from 9 of the positive clones were then probed with the internal probes neo, B, and C (FIG. 1(a) on different combinations of XbaI, XbaI-BamHI, and SalI digests to exclude concatamerization, inversions and uneven cross-over. Genomic DNA from tail biopsies of resulting mice was extracted using “Gitschier lysis buffer.” (Kogan et al., N. Engl. J. Med., 317:985-90 (1987)). The neo gene was present in all the chimeras but was efficiently excised in all male offspring (FIG. 1(b), in accordance with the Cre recombinase being expressed in the haploid germ cells of male chimeras.

Excision of the neo gene and presence of the Y719F-Kit mutation was further verified by PCR on genomic DNA using primers 54 and 59 surrounding the loxP sites, and primers 42 and 53 surrounding the mutated exon 15 (FIG. 1(c)). The size difference between +/+ and Y719F/Y719F was 87 bp as expected for one loxP site with the surrounding Xba linkers. The middle PCR band seen in DNA from Y719F/+ was due to formation of a heteroduplex between wild type and mutant PCR product, as shown by re-running the purified band after denaturation, as well as, by sequencing. For subsequent generations of mice, routine genotyping was done by PCR using primers 54 and 59.

### EXAMPLE 3

### Isolation of Primary Bone Marrow-Derived Mast Cells, Immunoprecipitation and Western Blotting

Bone marrow cells were collected by flushing the marrow cavity of femurs and mast cells were derived by selective growth for 6 weeks in IL-3-containing medium (Opti-Mem I; [Gibco BRL], 10% fetal bovine serum, 0.5 ng/ml recombinant murine IL-3 [R & D Systems, Inc.]). Medium was replaced daily and cells were transferred to new dishes to remove adherent cells, including macrophages and megakaryocytes. Immunoprecipitation, Western blotting and Akt in vitro kinase assays were done as described (Blume-Jensen et al., Curr. Biol., 8:779-82 (1998); Blume-Jensen et al., EMBO J., 12:4199-209 (1993)), using extracts from 9×10⁶ mast cells per lane. Briefly, cells were starved for 12 h in Opti-Mem I medium without IL-3 and containing only 0.5% serum, before stimulation with 100 ng/ml murine SCF (R & D Systems, Inc.) for 8 minutes at 37° C., where indicated. The mouse monoclonal antibodies U5 and U10 recognize the p85-Bcr domain. Kit was detected using an affinity-purified rabbit anti-serum against the C-terminus of human Kit, Kit-C1-affi (Blume-Jensen et al., EMBO J., 12:4199-209 (1993); Blume-Jensen et al., EMBO J., 10:4121-4128 (1991)), which also recognizes rodent Kit, or with an affinity-purified goat anti-serum against the C-terminus of mouse Kit, M-14 (Santa Cruz Biotechnology, Inc.). The rabbit anti-serum against Akt recognizes the C-terminus and the monoclonal antibody 4G10 (UBI) was used to detect phosphotyrosine.

### EXAMPLE 4

### Cell Proliferation, Apoptosis, Immunofluorescence and Alkaline Phosphatase Studies

Eight and 10 day old pups derived from Y719F/Y719F (female) x Y719F/+(male) crosses were injected intraperitoneally with 50 μg/g bodyweight of a 5 mg/ml aqueous solution of BrdU (Zymed Laboratories) and sacrificed after two hours. Testicles were dissected and tail biopsies were taken for genotyping. After fixation in 4% paraformaldehyde, testes were cryo-sectioned, and parallel sections were processed for either BrdU staining, TUNEL assay or immunofluorescence staining. BrdU incorporation was detected using a primary biotinylated mouse anti-BrdU monoclonal antibody. Detection was accomplished by incubating the antibody-bound samples with peroxidase-conjugated streptavidin, and diamino-benzidine was used for development. (Zymed Laboratories). Cells were counter-stained with hematoxylin. TUNEL staining was performed using the “In situ cell death detection kit, AP” from Boehringer Mannheim, along with nitroblue tetrazolium chloride and 5-bromo-4-chloro-3-indolyl phosphate as substrate for alkaline phosphatase.

Immunofluorescence staining was performed using the Kit-C1-affi ab (0.3 μg/ml) as primary antibody and CY-3-conjugated goat-anti rabbit Ig as secondary. Specificity was confirmed using the M-14 anti-Kit antibody from Santa Cruz. Nuclei were counter-stained with DAPI. Perfusion fixation, paraffin embedding and sectioning, and hematoxylin-eosin staining was performed on adult testes, ovaries and skin lumps from the lumbar region, according to standard procedures. Genital ridges were isolated and stained as described. (Cooke et al., Meth. Enzymol., 225:37-58 (1993)).

### EXAMPLE 5

### Analysis of Y719F Genetically Altered Mice

To ensure that disruption of PI 3′-kinase signaling from the Kit/SCF-R in mutant mice was obtained, primary mast cells were examined from bone marrow of +/+ and Y719F/Y719F age-matched mice. (FIGS. 1(d-h) and EXAMPLE 3). The mutant mast cells were found to be fully viable.

Mast cells were also examined from gene-targeted control mice (loxP/loxP) that were generated in parallel with the mutants. These control mice contained the loxP site but not the Y719F mutation. The Kit/SCF-R from Y719F/Y719F and control mice were found to be the same size and were expressed at comparable levels. (FIG. 1(d)). The Y719F-Kit receptor was also kinase active and autophosphorylated on tyrosine residues upon SCF stimulation. (FIG. 1(e)). Further, the regulatory p85 subunit of PI 3′-kinase was expressed at similar levels in mast cells from mutants and controls. (FIG. 1(f)). Although p85 co-precipitated with equivalent amounts of Kit/SCF-R from SCF-stimulated +/+ and loxP/loxP control cells, association with Kit/SCF-R was not detected in the Y719F/Y719F mutant mast cells. (FIG. 1(g)).

To determine the extent of indirect activation of PI 3′-kinase by the mutant receptor, assays were performed to detect SCF-induced Akt activation. (FIG. 1(h)). Activation of Akt, a serine/threonine kinase, is dependent on PI 3′-kinase and is known to be a sensitive measure of PI 3′-kinase activity. (Alessi and Cohen, Curr. Opin. Genet Dev., 8:55-62 (1998)). Although SCF activated Akt 5-fold in +/+ and loxP/loxP mast cells in a wortmannin-sensitive manner, Akt activation was only 12% of wild type levels in Y719F/Y719F mast cells. The minimal AKT activation in Y719F/Y719F mast cells was completely inhibited by wortmannin. Thus, from these experiments, it was apparent that PI 3′-kinase binding to the Kit receptor in the Y719F mutant was completely abolished and that SCF-induced PI 3′-kinase activation was reduced in mutant cells by approximately 90%.

Further analysis of the mutant mice revealed that heterozygous mice and homozygous mutant females were fully fertile but the Y719F/Y719F males were sterile (Table 1).

TABLE 1
BREEDING RESULTS

| | | | |
|---|---|---|---|
| Female: | +/+ | Y719F/+ | Y719F/Y719F |
| Male: | | | |
| +/+ | 7.8 +/− 0.32 (M) | 8.6 +/− 0.35 (M) | 8.3 +/− 0.28 (M) |
| | 6.3 +/− 0.6 (I) | 5.8 +/− 1.1 (I) | 6.4 +/− 0.7 (I) |
| Y719F/+ | 7.7 +/− 0.31 (M) | 7.6 +/− 0.28 (M) | 8.0 +/− 0.34 (M) |
| | 5.7 +/− 0.9 (I) | 6.2 +/− 1.2 (I) | 6.6 +/− 0.6 (I) |
| Y719F/Y719F | 0(M) | 0(M) | 0(M) |
| | 0(I) | 0(I) | 0(I) |

*Each number is the average litter size obtained from matings between B6D2F1/SV129 mice (‘M’; mixed background) or between SV129 mice (‘I’; inbred strain). Mean +/− S.E.M., n > 15 for ‘M’; n > 6 for ‘I’. Each mean represents breedings between a minimum of 7 males and 15 females for ‘M’ and 3 males and 6 females for ‘I’.

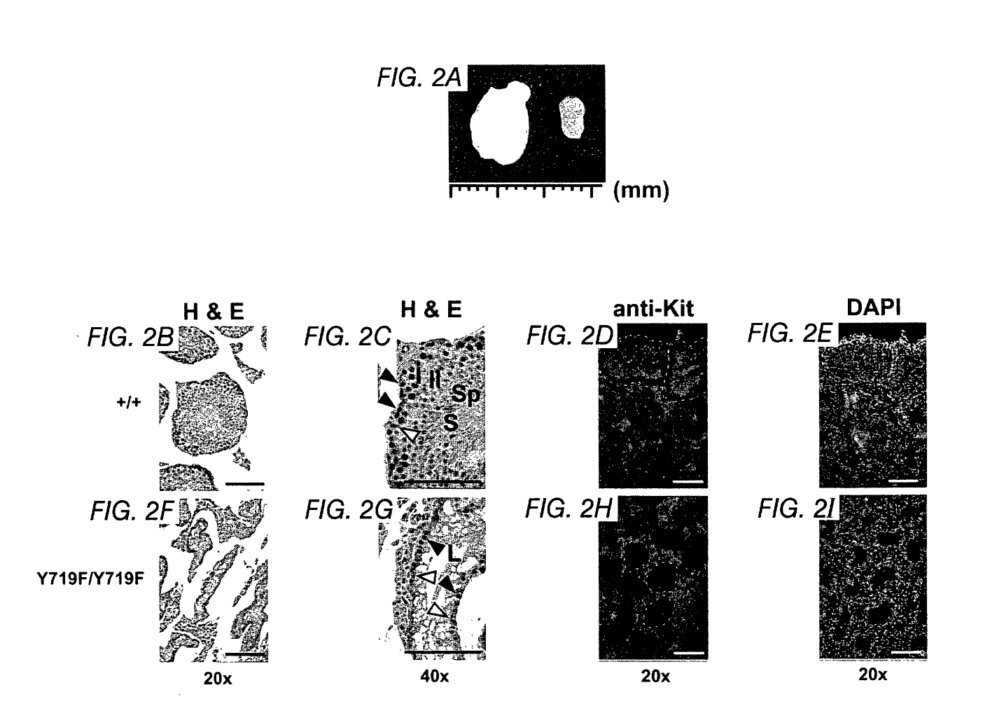

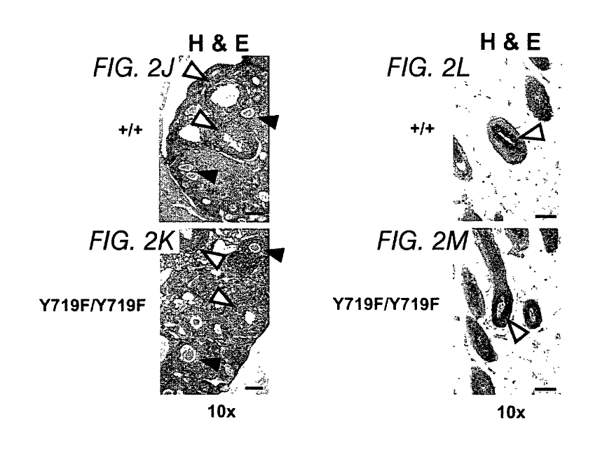

The male sterile phenotype was observed in both the ‘mixed background’ (B6D2F1 x 129/Sv) mice, as well as, inbred (129/Sv) mice. Mutant males, however, exhibited normal libido and produced vaginal plugs, despite having testes that were severely hypoplastic. (FIG. 2(a)).

The testes of mutant and control mice were more closely examined by dissection and microscopic analysis. Adult (4 months old) littermate mice and age-matched loxP/loxP control mice were perfusion-fixed and their testes were dissected. (FIGS. 2(b-i)). The seminiferous tubules of +/+ mice were observed to contain spermatogonial type A and B stem cells mixed with Sertoli cells near the basement lamina and primary and secondary spermatocytes, spermatids and spermatozoa most proximal to the lumen (FIGS. 2(b-c)). Kit/SCF-R was found to be strongly expressed in the single layer of outer spermatogonial cells and in secondary spermatocytes, spermatids and spermatozoa but Kit expression was absent in primary spermatocytes and Sertoli cells. (FIGS. 2(d-e)).

Moreover, Kit expression was observed in the Leydig cells of the stromal interstitial tissue of +/+ mice. In contrast, tubules of Y719F/Y719F mice contained only two distinct cell types, Sertoli cells and spermatogonial stem cells, in a single outer cell layer. Cellular debris filled the tubular lumina. (FIGS. 2(f-g)). Cells expressing Kit were not found in the seminiferous tubules of Y719F/Y719F mice, despite intense Kit staining of the testicular interstitium. (FIGS. 2(h-i)). These results provided evidence that the testicular hypoplasia and infertility of Y719F/Y719F males was due to an early spermatogonial stem cell differentiation block. Interestingly, although Kit/SCF-R has been implicated in oogenesis and is expressed on oocytes (Manova et al., Dev. Biol., 157:85-99 (1993)), ovaries of Y719F/Y719F females were of normal size and contained a normal number and size of oocytes and ruptured follicles, consistent with the intact fertility of female mutant mice. (FIGS. 2(j-k)).

Since Kit/SCF-R is also required for melanogenesis and hematopoiesis (Besmer, et al., Dev. Suppl., 125-37 (1993); Galli et al., Adv Immunol, 55:1-96 (1994); Lyman and Jacobsen, Blood, 91:1101-34 (1998)), pigmentation and blood parameters were then examined in the genetically altered mice. The Y719F/Y719F mice were found to have normal skin and retinal pigmentation. Further, the total number of melanocytes at the base of hair follicles of the mutant mice were found to be normal. (FIGS. 2(l-m)). Similarly, all of the hematological parameters examined, including white cell and differential counts, revealed concordant profiles among littermate Y719F/Y719F and +/+ mice (Table 2), as well as, loxP/loxP control mice.

In particular, hemoglobin (Hgb), mean cell volume (MCV), and mean cell hemoglobin concentration (MCHC) were all normal in the mutant mice. These results provided evidence that the mutant mice do not suffer from macrocytic anemia. Macrocytic anemia is an aspect of the murine W and human piebald phenotypes, both of which are due to naturally occurring loss-of-function mutations in Kit/SCF-R. Moreover, an increased number of immature precursors or blast cells was not detected in smears from bone marrow or peripheral blood.

TABLE 2
BLOOD PARAMETERS

| | Genotype: | | | |
|---|---|---|---|---|
| | +/+ (M) | Y719F/Y719F (M) | +/+ (I) | Y719F/Y719F (I) |
| | (mean +/− S.E., n = 8) | (mean +/− S.E., n = 8) | (mean, n = 4) | (mean, n = 4) |
| RBC (×10⁶/μl) | 10.0 +/− 0.5 | 9.13 +/− 1.0 | 9.8 | 9.6 |
| hgb (g/dl) | 15.1 +/− 0.8 | 14.5 +/− 1.3 | 15.5 | 15.6 |
| Hct (%) | 48.3 +/− 2.8 | 45.6 +/− 3.8 | 48.7 | 49 |
| MCV (fl) | 48.0 +/31 2.3 | 45.6 +/− 3.8 | 50.5 | 51.3 |
| MCHC (g/dl) | 31.3 +/− 1.1 | 31.7 +/− 0.8 | 31.8 | 31.8 |
| RDW (%) | 19.8 +/− 1.8 | 19.5 +/− 2.1 | 19.3 | 20.2 |
| Plat (×10³/μl) | 1149 +/− 286 | 952 +/− 212 | 830 | 817 |
| MPV (fl) | 6.8 +/− 0.4 | 6.8 +/− 0.3 | 6.1 | 5.7 |
| WBC (×10³/μl) | 7.0 +/− 1.5 | 8.4 +/− 2.2 | 8.9 | 9.3 |
| Neu (% N) | 18.3 +/− 4.9 | 24.7 +/− 4.7 | 17 | 28 |
| Lymph (% L) | 78.7 +/− 6.2 | 73.3 +/− 4.5 | 68.5 82.5 | |
| Mono (% M) | 2.5 +/− 1.9 | 1.2 +/− 1.0 | 4 | 0.5 |
| Eos (% E) | 0.5 +/− 0.5 | 1.6 +/− 1.6 | 0 | 0.5 |
| Baso (% B) | N.D. | N.D. | N.D. | N.D. |
| *Peripheral blood obtained by retro-orbital bleedings was subjected to automated complete blood cell counts, platelet counts, and differential counts as well as manual differential counts on blood smears. ‘M’ is ‘mixed’ background mice, B6D2F1/SV129. ‘I’ is ‘inbred’ strain mice, SV129. Mean +/− S.E. (sample standard error, σₙ₋₁, where n = 8) for ‘M’, mean (n = 4) for ‘I’. RBC, red blood cells; Hgb, hemoglobin; Hct, hematocrit; | | | | |
| # MCV, mean red cell volume; MCHC, mean cell hemoglobin concentration; RDW, red cell distribution width; plat, platelets; MPV, mean platelet volume; WBC, white blood cells; Neu, neutrophils; Lymph, lymphocytes; Mono, monocytes; Eos, eosinophils; Baso, basophils. | | | | |

N.D., not detected.

Next, an extensive expression analysis by flow cytometry of the cell surface differentiation markers CD3ε, CD4, CD5, CD8a, CD24 (HSA), CD25 (IL2-Rα), CD44, CD45 (B220), Gr-1, Mac-1 and CD117 (c-Kit) was performed on cells from thymus, spleen and bone marrow of 5 weeks old wild type and mutant animals. The number of cells expressing each marker and the expression mean of marker-positive cells were found to be virtually identical in wild type and mutant cells. In particular, Kit expression levels were found to be equivalent in Y719F/Y719F mice. These data established that SCF-induced PI 3′-kinase signaling was not essential for melanogenesis or to sustain normal hematopoiesis of non-challenged mice.

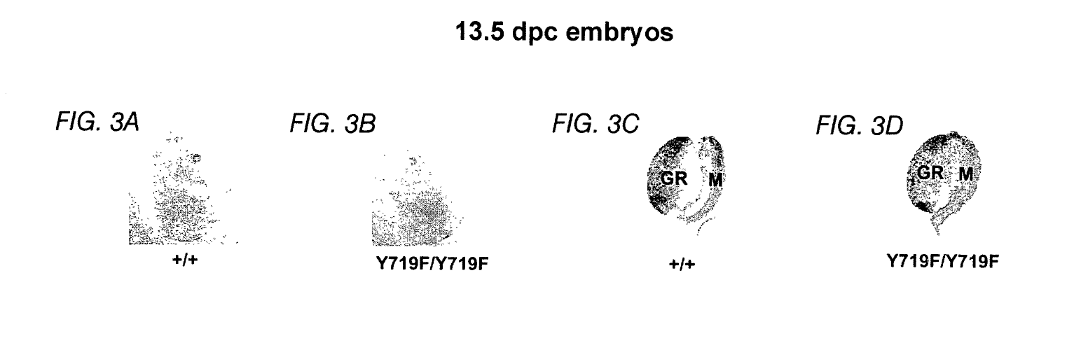

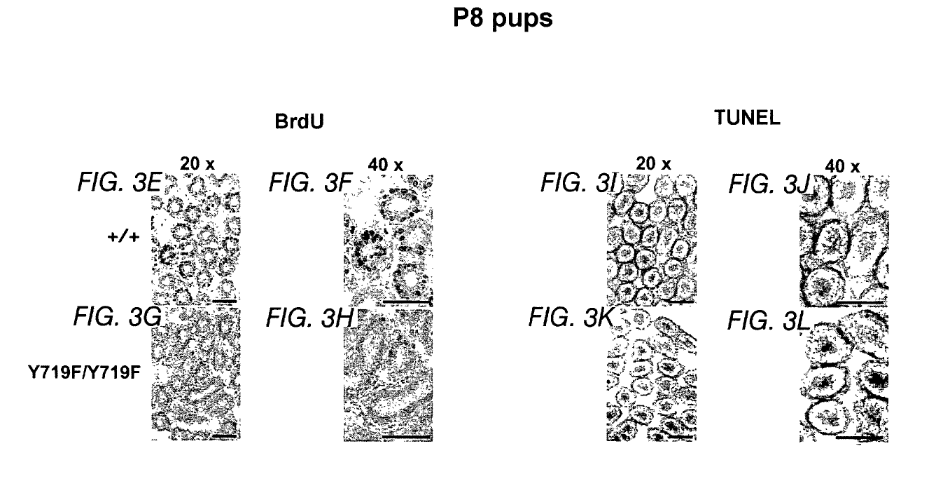

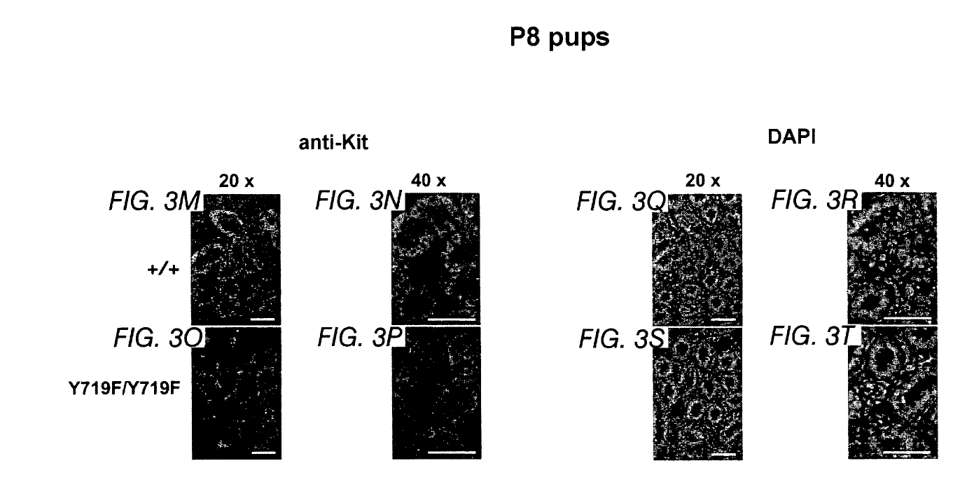

To exclude the possibility that the Y719F mutation caused a major pre-natal defect(s) in development and migration of primordial germ cells, isolated genital ridges from Y719F/Y719F and +/+ littermate embryos were examined at 13.5 days post coitus (dpc). Primordial germ cells (PGC) were observed to enter the genital ridges at 11.5 dpc and were easily detected by alkaline phosphatase staining until ˜14.5 dpc, at which time the definitive gonads begin developing. (Cooke et al., Meth. Enzymol., 225:37-58 (1993)). The developing gonads of Y719F/Y719F and +/+ embryos were of similar size, and alkaline phosphatase-stained whole mounts revealed that PGC were present not only in wild type but also mutant animals. (FIGS. 3(a-d)). These results together with the analysis of adult testes provided above established that mutant males have a major defect in spermatogonial stem cell differentiation.

Testes from post-natal day 8 (P8) and P10 pups were then examined to determine more whether the Y719F mutation caused a defect in spermatogonial stem cell differentiation. Spermatogonial cells typically begin to express Kit/SCF-R at approximately P7. Only spermatogonial and Sertoli cells are present in P8 pups, while preleptotene spermatocytes begin to appear in the testes at P10.

In P8 pups the organization, morphology and cell types of seminiferous tubules from mutant mice were indistinguishable from those of wild type mice. (FIGS. 3(e-t)). However, in vivo BrdU labeling of P8 pups demonstrated that germ cell proliferation was decreased in Y719F/Y719F mice compared to +/+ litters. (FIGS. 3(e-h) and see also EXAMPLE 4). Apoptotic cells were not observed in either +/+ or homozygous mutant mice at this stage. (FIGS. 3(i-l)). In mutant and wild type testes the distribution of Kit immuno-positive cells in parallel sections was similar to that of BrdU-positive cells, consistent with previous reports that proliferating spermatogonial cells are Kit-positive. (Manova et al., Development, 110:1057-69 (1990); Schrans-Stassen et al., Endocrinology, 140:5894-5900 (1999)) (compare FIGS. 3(m-p) with FIGS. 3(e-h)). Accordingly, fewer Kit-positive cells were observed in mutant seminiferous tubules (FIGS. 3(o-p) despite the presence of cells in all tubules. (FIGS. 3(q-t)).

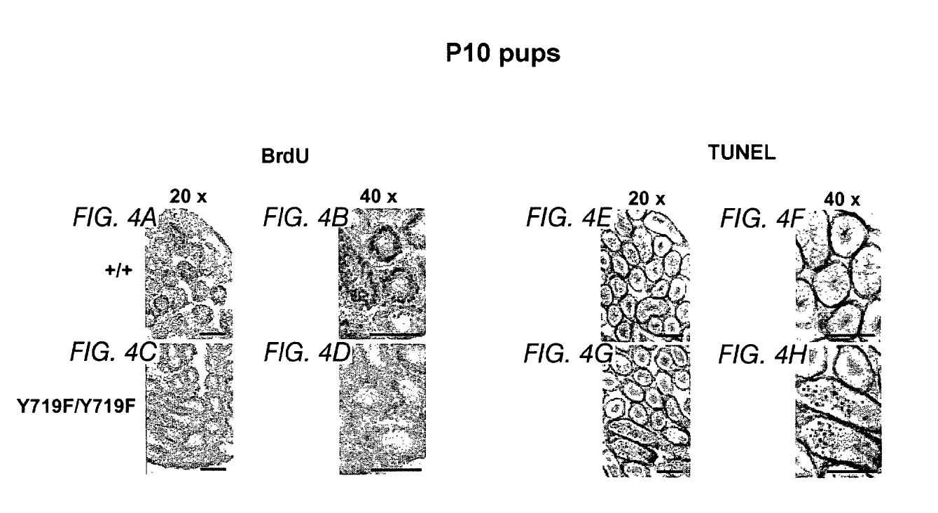

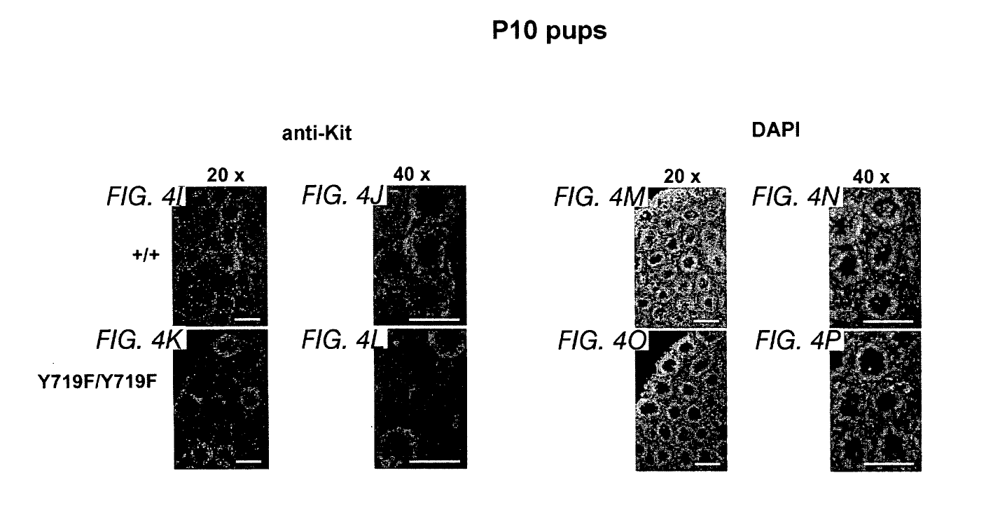

In P10 pups a drastic reduction in germ cell proliferation was observed (FIGS. 4(a-d)). Interestingly, at this stage the tubules from Y719F/Y719F mice still contained only spermatogonial and Sertoli cells organized in two outer cell layers, while the tubules from +/+ mice were organized in 3-4 cell layers containing primary spermatocytes, as well. (FIG. 4). Extensive apoptosis was observed in some tubules from the P10 Y719F/Y719F mice. (FIGS. 4(e-h)). While the outer layer of germ cells in +/+ mice was Kit-positive (FIGS. 4(i-l)), the majority of tubules in P10 mutant pups did not express Kit/SCF-R (FIGS. 4(k-l)), despite the presence of one or two outer cell layer(s) (FIGS. 4(o-p)). These cells are morphologically indistinguishable from the Sertoli cells and more undifferentiated, Kit-negative spermatogonia seen in adult mutants. These results provided evidence that testicular development in mutant mice was not perturbed until P8, since the testes are normally organized and contain the different cell types, including germ cells, at roughly normal numbers. Only few Kit-positive spermatogonial cells were generated in mutant testes at this stage, however. Eventually these cells underwent apoptosis, leaving only Kit-negative, more undifferentiated spermatogonial stem cells behind together with Sertoli cells. (FIGS. 2(f-i)).

Although the invention has been described with reference to embodiments and examples, it should be understood that various modifications can be made without departing from the spirit of the invention. Accordingly, the invention is limited only by the following claims.

What is claimed is:

1. A genetically altered mouse carrying a modification of a Kit/Stem cell factor receptor gene in both alleles, wherein the modification results in sterility of the mouse, but does not result in other major phenotypic alterations in the mouse.

2. The genetically altered mouse of claim 1, wherein said modification comprises a codon substitution at amino acid position 719.

3. The genetically altered mouse of claim 2, wherein said modification comprises a phenylalanine substitution at amino acid position 719.

4. The genetically altered mouse of claim 1, wherein said Kit/Stem cell factor receptor gene comprises the nucleic acid sequence of SEQ ID NO: 1, wherein a tyrosine codon that encodes amino acid position 719 is modified encode a phenylalanine amino acid.

5. The genetically altered mouse of claim 1, wherein the Kit/Stem cell factor receptor gene encodes a protein comprising the amino acid sequence of SEQ ID NO: 2 that is modified at amino acid position 719 to comprise a Y719F substitution.

6. The genetically altered mouse of claim 1, wherein said spermatogenesis is reduced to undetectable levels.

7. A method for producing a genetically altered mouse exhibiting reduced levels of spermatogenesis, comprising:
(a) providing a modified Kit/Stem cell factor receptor gene targeting construct comprising a Kit/Stem cell factor receptor gene having a modification resulting in a codon substitution of a tyrosine residue at amino acid position 719;
(b) introducing said modified Kit/Stem cell factor receptor gene and a selectable marker sequence into a mouse embryonic stem cell;
(c) introducing said mouse embryonic stem cell into a mouse embryo;
(d) transplanting said embryo into a pseudopregnant mouse;
(e) allowing said embryo to develop to term;
(f) identifying a genetically altered mouse whose genome comprises a modification of the endogenous Kit/Stem cell factor receptor gene in both alleles; and
(g) breeding the genetically altered mouse of step (f) to obtain a genetically altered mouse whose genome comprises a modification of the endogenous Kit/Stem cell factor receptor gene, wherein said disruption results in said mouse exhibiting reduced levels of spermatogenesis as compared to a wild-type mouse.

8. The genetically altered mouse of claim 1, wherein said modification comprises a phenylalanine substitution at amino acid position 719.

9. An antibody that specifically binds to an isolated murine Kit/Stem cell factor receptor polypeptide comprising an amino acid substitution at amino acid position 719, but does not specifically bind to a wild-type murine Kit/Stem cell factor receptor polypeptide.

10. The antibody of claim 9, wherein the antibody is a monoclonal antibody.

11. An antibody that specifically binds to an isolated human Kit/Stem cell factor receptor polypeptide comprising an amino acid substitution at amino acid position 721, but does not specifically bind to a wild-type human Kit/Stem cell factor receptor polypeptide.

12. The antibody of claim 11, wherein the antibody is a monoclonal antibody.

13. A method of identifying a fertility defect in a male comprising:
identifying a male at risk for a fertility defect; and
detecting the presence of a non-phosphorylated tyrosine residue in the kinase insert region of a Kit/Stem cell factor receptor in said male, wherein the presence of such non-phosphorylation is indicative of a fertility defect.

14. A method of identifying a fertility defect in a male human comprising:
identifying a male at risk for a fertility defect;
detecting the presence of a mutation in a nucleic acid sequence encoding a Kit/Stem cell factor receptor gene in said male, wherein said mutation results in an amino acid substitution at position 721 of the Kit/Stem cell factor receptor polypeptide.

15. A method of detecting a fertility defect in a male human, comprising:
obtaining from the male human a biological sample containing polynucleotides; and
identifying the presence of a polynucleotide encoding a Kit/Stem cell factor receptor protein, wherein said polynucleotide comprises a mutation in a kinase insert region, and wherein said mutation results in a substitution or deletion of a tyrosine amino acid within said region, and wherein the presence of said mutation is indicative of a fertility defect in said male human.

16. A computer-based system for identifying an agent that interacts with a kinase insert region of a wild type or mutant Kit/Stem cell factor receptor protein comprising:
a first database comprising data representing the kinase insert region of a wild type or mutant Kit/Stem cell factor receptor protein;
a second database comprising data representing potential compounds that bind with said kinase insert region; and
a search program comprising instructions that, when executed, determine potential compounds that bind to said kinase insert region.

17. The system of claim 16, wherein said compounds are selected from the group consisting of a peptide, a peptidomimetic, and a chemical.

18. The system of claim 16, wherein said data representing said kinase insert region comprises the three-dimensional structure of said kinase insert region.

19. The system of claim 16, wherein said data representing said potential compounds comprises-the three-dimensional structure of said potential compounds.

20. A method for identifying an agent that interacts with a kinase insert region of a wild type or mutant Kit/Stem cell factor receptor protein comprising:
providing data representing a kinase insert region of a wild type or mutant Kit/Stem cell factor receptor protein;
providing data representing a potential compound that might bind to said kinase insert region; and
aligning the data representing the kinase insert region of the wild type or mutant Kit/Stem cell factor receptor protein with said data representing said potential compound to identify an interaction between said kinase insert region and said potential compound.

21. The method of claim 20, wherein said compound is selected from the group consisting of a peptide, a peptidomimetic, and a chemical.

22. The method of claim 20, wherein said data representing said kinase insert region comprises the three-dimensional structure of said kinase insert region.

23. The method of claim 20, wherein said data representing said potential compound comprises the three-dimensional structure of said potential compound.

24. A method of identifying an agent that interacts with the kinase insert region of a wild type or mutant Kit/Stem cell factor receptor protein comprising:
providing a cell comprising a nucleic acid encoding a kinase insert region of a wild type or mutant Kit/Stem cell factor receptor protein;
contacting said cell with the agent; and
detecting an interaction between the kinase insert region of a wild type or mutant Kit/Stem cell factor receptor protein and the agent.

25. A method of identifying an agent that interacts with a kinase insert region of a wild type or mutant Kit/Stem cell factor receptor protein:
providing a multimeric support having the kinase insert region of a wild type or mutant Kit/Stem cell factor receptor protein;
contacting the multimeric support with the agent; and
detecting an interaction of the agent with the kinase insert region of a wild type or mutant Kit/Stem cell factor receptor protein on the multimeric support.

26. A pharmaceutical composition that inhibits male reproduction, comprising an agent that interacts with a kinase insert region of a Kit/Stem cell factor receptor protein.

27. The composition of claim 26, wherein said Kit/Stem cell factor receptor is a human Kit/Stem cell factor receptor and said composition inhibits phosphorylation of a tyrosine amino acid at position 721.

28. The composition of claim 27, wherein said composition comprises a peptidomimetic.

29. The composition of claim 28, wherein said composition comprises an antibody.

30. A method of inhibiting spermatogenesis comprising the steps of:
identifying a male mammal in need of a compound that inhibits spermatogenesis; and
administering to said male mammal a therapeutically sufficient amount of a compound having the formula:
X¹ₙpYMX²X³MX⁴ₙ,
wherein:
“X¹ₙ” and “X⁴” comprise any amino acid residue;
“n” comprises a number between 1-1000 residues;
“pY” comprises phosphorylated tyrosine;
“M” comprises methionine; and
X² comprises an amino acid selected from the group consisting of: methionine, valine, isoleucine, and glutamine.


SEQUENCE LISTING (6 sequences)

SEQ ID NO: 1 - DNA, 5098 bp, MUS MUSCULUS
gagctcagagtctagcgcagccaccgcgatgagaggcgctcgcggcgcctgggatctgctctgcgtcctgttggtcctgctccgtggccagacagccacgtctcagccatctgcaagtccaggggagccgtctccgccatccatccatccagcacaatcagagttaatagttgaagctggcgacaccctcagcctgacgtgcattgatcccgactttgtcagatggactttcaagacctatttcaatgaaatggttgagaataaaaaaaatgaatggatccaggaaaaagccgaggccactcgcacgggcacatacacgtgcagcaacagcaatggcctcacgagttctatttacgtgtttgttagagatcctgccaaacttttcctggttggccttcccttgtttggcaaagaagacagcgacgcgctggtccgctgccctctgacagacccacaggtgtccaattattccctcatcgagtgtgatgggaaatctctccccacggacctgacgtttgtcccaaaccccaaggctggcatcaccatcaaaaacgtgaagcgcgcctaccaccggctctgtgtccgctgtgctgctcagcgtgacggtacatggctgcattctgacaaattcaccctcaaagtgcgggaagccatcaaggctatccctgttgtgtctgtgcctgaaacaagtcacctccttaagaaaggggacacatttacggtggtgtgcaccataaaagatgtgtctacatccgtgaactccatgtggctaaagatgaaccctcagcctcagcacatagcccaggtaaagcacaatagctggcaccggggtgacttcaattatgaacgccaggagacgctgactatcagctcggcaagagttgacgattctggagtgttcatgtgttatgccaataatacttttggatcagcaaatgtcacaacaaccttgaaagtagtagaaaaaggattcatcaacatctcccctgtgaagaacactacagtatttgtaaccgatggagaaaacgtagatttggttgttgaatacgaggcctaccccaaacccgagcaccagcagtggatatatatgaacaggacctcggctaacaaagggaaggattatgtcaaatctgataacaaaagcaacatcagatatgtgaaccaacttcgcctgaccagattaaaaggcacagaaggaggcacttatacctttctggtgtccaactctgatgccagtgcttccgtgacattcaacgtttacgtgaacacaaaaccagaaatcctgacgtacgacaggctcataaatggcatgctccagtgtgtggcagagggattcccggagcccacaatagattggtatttttgtacaggagcagagcaaaggtgtaccactcctgtctcaccagtggacgtacaggtccagaatgtatctgtgtcaccatttggaaaactggtggttcagagttccatagactccagcgtcttccggcacaacggcacggtggagtgtaaggcctccaacgatgtgggcaagagttccgccttctttaactttgcatttaaagagcaaatccaggcccacactctgttcacgccgctgctcattggctttgtggtcgcagctggcgcgatggggatcattgtgatggtgctcacctacaaatatttgcagaaacccatgtatgaagtacaatggaaggttgtcgaggagataaatggaaacaattatgtttacatagacccgacgcaacttccttatgatcacaaatgggagtttcccagaaacaggctgagttttggaaagacattgggagctggtgccttcgggaaggtcgttgaggccactgcatatggcttgattaagtcggatgctgccatgacagttgccgtgaagatgctcaaaccaagtgcccatttaacagaaagagaggccctaatgtcggaactgaaggtcctgagctacctgggcaatcacatgaatattgtgaacctgcttggcgcatgcacggtgggagggcccaccctggtcattacagaatattgttgctatggtgatcttttgaattttttgagaaggaagcgtgactcgtttattttctcaaagcaagaagagcaggcagaagcggcactttataagaaccttctgcactcaacggagccttcctgtgacagttcaaatgaatatatggacatgaagcctggcgtttcctacgtggtgccaaccaagacagacaagaggagatccgcaagaatagactcgtacatagaaagagacgtgactcctgccatcatggaagatgacgagctggctctggacctggatgatttgctgagcttctcctaccaggtggccaaggcgatggcgttcctcgcctccaagaattgtattcacagagatttggcagccaggaatatcctcctcactcacgggcggatcacaaagatttgcgatttcgggctagccagagacatcaggaatgattcgaattacgtggtcaaaggaaatgcacgactgcccgtgaagtggatggcaccagagagcattttcagctgcgtgtacacatttgaaagtgatgtctggtcctatgggattttcctctgggagctcttctccttaggaagcagcccctacccagggatgccggtcgactccaagttctacaagatgatcaaggaaggcttccggatggtcagcccggagcacgcgcctgccgaaatgtatgacgtcatgaagacttgctgggacgctgaccccttgaaaaggccaacattcaagcaggttgtccaacttattgagaagcagatctcggacagcaccaagcacatttactccaacttggcaaactgcaaccccaacccagagaaccccgtggtggtggaccattccgtgagggtcaactcggtgggcagcagcgcctcttctacgcagcccctgctcgtgcacgaagatgcctgagcagaaacccaagtccaacaggctttgctgctgtctccgaccccgtccttctggcttctgtgatggttacttggtttccctttgacttgcatcctattccagggtagcgagttccccaccccacctccaaccccactgtgattccgcctttacgagcacacactttagtggccgatggcttttcttttctgccatcagccaccgtcccgctgcgaaggtccgaactgtatgtatatattttcccaatagcaaagtagctcctactgtaaacagaaggactcctcctgctttagaggagaagggaagggcggggtgaaactggatgcccagagttcttcccccagtgctcccctgagtgtatttgaaaagtatggccagtagttcacttgaagaatagatgtagtcccatttggccctgagagccatccttaatgatgggagatatatgtagcaagactagaaagccaagccctttgtgtagaaagcagaccattcttagaacagagggcaacggggcatcggaagtctggtcacgctaagaagaccgaggctgagaaggaacaagccaggggaagcgtgaacaatgatgctctgctctgggctgccgctcgggcttctgtacaactgacctggtttctcagtactttgctgtctgggagtagcattggaatcaaggcctcctccctagtcagcctttgtatatactcatctatacgttgtatgcgttcatactttggaggagggatttcccacaagctttcgtttctgtgtacagccctggattagacctactgtgtgtaagaatagattaagagccatacatatttgaaggaaacagttaaatgttttttggttgtggttgttgttgttgttgttttaaagaaaaaaatgtatatgctaagcacaatctttataagacctcttagccaacatacttgctctgtctacacttcggaacaagccttccatgtcagagtggctttgcaggcaggagaactgaggctgtttgaaaaggttaccacaggatggagaaaacagtgcagtcctggtttggattctcacatagcagggagcacaagttaaactcgaccttttataggcacgtcccggacatcgggcctgtatctattcaagtgtgtatgtgtgtgcatgcgtgtgtctatgcgtgtgggtgagttgtgttgggaaacttgccctgcatccctgagggtcctccttcaggacccaagacgtaacagcttctgtcaccgctcctgtctctccagtttccctgcatgtcgctcactgtctagaatttactcaaagccgccacagaggcttagcggagtgaagtgccgaaggacctctttatttggagtcctcctgtatttaacaacactcttatcgtagacccattcattagaccttatgtaatgctgccaatccagggaaacagatttaaagtgtaccccgtagacagggcccagaggttcccttgtccttgccctcccccacaccacccatgatcactgtccaacataaagggttcagtgtgttacgtggtcatgtgttgtccttacaggattcaggtatgttgccttcacggttttccccaccccctcctgccctttatcctttaggccgtgtggccatgaacctggaagaagtgatcgtttcgacttgagtgctacactcttgcacctttccaaagtaagctggtttggaggtcctgtggtcatgtacgagactgtcaccagttaccgcgctctgtttgaaacatgtctttgtattcctaatgacttcagttagagtaaggagaatagctgttaatatggatgtcaggtacttaaggggccacaccattgagaattttgtcttggatattcttgaaagtttatatttttataattttttttacatcagatgtcagatgtttctttcagttgcttgatgtttggaattattatgtggctttttttgtaaatattgaaatgtagcaataatgtcttttgaatattcctgagcccatgagtccctgaaaatattttttatatatacagtaactttatgtgtaaataatacgctgtgcaagtttaaacatgtcacgttacatgtgggttttttctgatatgttgtccaactgttgacagttctgaagaattc

SEQ ID NO: 2 - protein, 975 aa, MUS MUSCULUS
MRGARGAWDLLCVLLVLLRGQTATSQPSASPGEPSPPSIHPAQSELIVEAGDTLSLTCIDPDFVRWTFKTYFNEMVENKKNEWIQEKAEATRTGTYTCSNSNGLTSSIYVFVRDPAKLFLVGLPLFGKEDSDALVRCPLTDPQVSNYSLIECDGKSLPTDLTFVPNPKAGITIKNVKRAYHRLCVRCAAQRDGTWLHSDKFTLKVREAIKAIPVVSVPETSHLLKKGDTFTVVCTIKDVSTSVNSMWLKMNPQPQHIAQVKHNSWHRGDFNYERQETLTISSARVDDSGVFMCYANNTFGSANVTTTLKVVEKGFINISPVKNTTVFVTDGENVDLVVEYEAYPKPEHQQWIYMNRTSANKGKDYVKSDNKSNIRYVNQLRLTRLKGTEGGTYTFLVSNSDASASVTFNVYVNTKPEILTYDRLINGMLQCVAEGFPEPTIDWYFCTGAEQRCTTPVSPVDVQVQNVSVSPFGKLVVQSSIDSSVFRHNGTVECKASNDVGKSSAFFNFAFKEQIQAHTLFTPLLIGFVVAAGAMGIIVMVLTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKAMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 3 - DNA, 5084 bp, HOMO SAPIENS
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 4 - protein, 976 aa, HOMO SAPIENS [sequence omitted]

SEQ ID NO: 5 - protein, 11 aa, Artificial Sequence
  Artificial Peptide
TNEYMDMKPEV

SEQ ID NO: 6 - protein, 8 aa, Artificial Sequence
  Artificial Peptide
YVPMLDMK